(19) United States
(12) Patent Application Publication
(10) Pub. No.: US 2007/0264285 A1
(43) Pub. Date: Nov. 15, 2007

(54) Compositions Comprising Cationic Microparticles And Hcv B1e2 Dna And Methods Of Use Thereof

(76) Inventors: Derek O'Hagan, Berkeley, CA (US); Michael Houghton, Danville, CA (US); Manmohan Singh, San Ramon, CA (US)

Correspondence Address: NOVARTIS VACCINES AND DIAGNOSTICS INC., INTELLECTUAL PROPERTY R338, P.O. BOX 8097, Emeryville, CA, 94662-8097, US

(21) Appl. No.: 10/554,625

(22) PCT Filed: Apr. 23, 2004
(86) PCT No.: PCT/US04/12510

§ 371 Date: Mar. 21, 2007

Related U.S. Application Data
(60) Provisional application No. 60/465,841, filed on Apr. 25, 2003.

(51) Int. Cl. A61K 39/29; A61P 31/12

(52) U.S. Cl. 424/228.1

(57) ABSTRACT

Immunogenic HCV E1E2₈₀₉ DNA compositions and methods of using the same are described. The compositions comprise HCV EIE2₈₀₉ DNA adsorbed to cationic microparticles, such as poly(lactide-co-glycolide) (PLG) microparticles.

### TECNICAL FIELD

The present invention pertains generally to immunogenic compositions comprising DNA encoding HCV immunogens. In particular, the invention relates to compositions comprising DNA encoding HCV E1E2 polypeptides adsorbed to cationic microparticles and methods of using the same.

### BACKGROUND

Hepatitis C virus (HCV) was identified over a decade ago and is now known to be the leading cause of non-A and non-B viral hepatitis (Choo et al., Science (1989) 244:359-362; Armstrong et al., Hepatology (2000) 31:777). HCV infects approximately 3% of the world population, an estimated 200 million people (Cohen, J., Science (1999) 285:26). About 30,000 newly acquired HCV infections occur in the United States annually. Additionally, there is a large incidence of HCV infection in developing countries. Although the immune response is capable of clearing HCV infection, the majority of infections become chronic. Most acute infections remain asymptomatic and liver disease usually occurs only after years of chronic infection.

The viral genomic sequence of HCV is known, as are methods for obtaining the sequence. See, e.g., International Publication Nos. WO 89/04669; WO 90/11089; and WO 90/14436. HCV has a 9.5 kb positive-sense, single-stranded RNA genome and is a member of the Flaviridae family of viruses. At least six distinct but related genotypes of HCV, based on phylogenetic analyses, have been identified (Simmonds et al., J. Gen. Virol. (1993) 74:2391-2399). The virus encodes a single polyprotein having more than 3000 amino acid residues (Choo et al., Science (1989) 244:359-362; Choo et al., Proc. Natl. Acad. Sci. USA (1991) 88:2451-2455; Han et al., Proc. Natl. Acad. Sci. USA (1991) 88:1711-1715). The polyprotein is processed co- and post-translationally into both structural and non-structural (NS) proteins. Two of the structural proteins are envelope glycoproteins known as E1 and E2. The HCV E1 and E2 glycoproteins have been shown to be protective against viral challenge in primate studies. (Choo et al., Proc. Natl. Acad. Sci. USA (1994) 91:1294-1298).

Currently, the only available therapies for HCV are IFN-α and ribavirin. Unfortunately, these agents are effective in less than half the patients treated (Poynard et al., Lancet (1998) 352:1426; McHutchison et al., Engl. J. Med. (1998) 339:1485). Therefore, there is an urgent need for the development of efficacious vaccines to prevent HCV infection, as well as for immunotherapies to be used as an alternative, or in conjunction with existing therapies.

T cell immunity to HCV may determine the outcome of HCV infection and disease (Missale et al., J. Clin. Invest. (1996) 98:706; Cooper et al., Immunity (1999) 10:439; and Lechner et al., J. Exp. Med. (2000) 191:1499). One study concluded that individuals displaying predominant Th0/Th1 CD4+T helper responses resolved their HCV infections, while those with Th2-type responses tended to progress to chronicity (Tsai et al., Hepatology (1997) 25:449458). In addition, it has been shown that there is an inverse correlation between the frequency of HCV-specific cytotoxic T lymphocytes (CTLs) and viral load (Nelson, et al., J. Immunol. (1997) 158:1473). Recently, control of HCV in chimpanzees was shown to be associated with a Th1 T cell response (Major et al., J. Virol. (2002) 76:6586-6595). Therefore, HCV-specific T cell responses appear to play an important role in controlling HCV infection. A role for antibodies in protection has also been proposed based on rare cases of spontaneous resolution of chronic infection in patients (Abrignani et al., J. Hepatol. (1999) 31 Supp11:259-263). Additionally, protection in primates has been associated directly with the titer of anti-E1E2 antibodies, evidencing a possible role for antibodies in protection (Choo et al., Proc. Natl. Acad. Sci. USA (1994) 91:1294-1298).

DNA vaccines have been shown to induce potent long-term CTL and Th1 cellular responses in a range of animal models (Gurunathan et al., Ann. Rev. Immunol. (2000) 18:927-974). Although DNA vaccines have been administered to human volunteers in a number of clinical trials and appear safe, their potency has been low relative to the responses achieved in smaller animal models (Gurunathan et al., Ann. Rev. Immunol. (2000) 18:927-974). For example, although detectable CTL responses have been induced in human volunteers, even high doses of DNA (2.5 mg) have sometimes failed to induce detectable antibody responses (Wang et al., Science (1998) 282:476480). Antibody responses were not detected in human volunteers even when a needle-free jet injection device was used for DNA delivery in an attempt to improve potency (Epstein et al., Hum. Gen. Ther (2002) 13:1551-1560). Hence, there is a clear need for improving the potency and efficacy of DNA vaccines, particularly for humoral responses.

Particulate carriers with adsorbed or entrapped antigens have been used in attempts to elicit adequate immune responses. Examples of particulate carriers include those derived from polymethyl methacrylate polymers, as well as microparticles derived from poly(lactides) (see, e.g., U.S. Pat. No. 3,773,919), poly(lactide-co-glycolides), known as PLG (see, e.g., U.S. Pat. No. 4,767,628) and polyethylene glycol, known as PEG (see, e.g., U.S. Pat. No. 5,648,095). Polymethyl methacrylate polymers are nondegradable while PLG particles biodegrade by random nonenzymatic hydrolysis of ester bonds to lactic and glycolic acids which are excreted along normal metabolic pathways.

Such carriers present multiple copies of a selected macromolecule to the immune system and promote trapping and retention of the molecules in local lymph nodes. The particles can be phagocytosed by macrophages and can enhance antigen presentation through cytokine release. International Publication No. WO 00/050006 describes the production of cationic microparticles with adsorbent surfaces. The use of cationic microparticles as a delivery system for DNA vaccines has been shown to dramatically improve potency (Singh et al., Proc. Natl. Acad. Sci. USA (2000) 97:811-816). For example, microparticles have been shown to enhance both humoral and T cell responses in a range of animal models when delivered in combination with plasmids encoding HIV antigens (Singh et al., Proc. Natl. Acad. Sci. USA (2000) 97:811-816; Briones et al., Pharm. Res. (2001) 18:709-712; O'Hagan et al., J. Virol. (2001) 75:9037-9043).

A number of studies have been undertaken to determine the mechanism of action for cationic PLG microparticles to induce enhanced responses to adsorbed DNA. Preliminary studies have shown that PLG/DNA, but not plasmid DNA is able to mediate transfection of dendritic cells in vitro (Denis-Mize et al, Gene Ther. (2000) 7:2105-2112). In addition, PLG/DNA protects DNA against degradation and enhances gene expression in muscle and local lymph nodes (Singh et al., Proc. Natl. Acad. Sci. USA (2000) 97:811-816; Briones et al., Pharm. Res. (2001) 18:709-712; Denis-Mize et al, Gene Ther. (2000) 7:2105-2112).

Despite the use of such particle delivery systems, conventional vaccines often fail to provide adequate protection against the targeted pathogen. Accordingly, there is a continuing need for effective immunogenic compositions against HCV which include safe and non-toxic delivery agents.

### SUMMARY OF THE INVENTION

The present invention is based in part, on the surprising discovery that the use of HCV E1E2₈₀₉ DNA, adsorbed to cationic microparticles, produces significantly higher antibody titers than those observed with E1E2 DNA alone. Cationic microparticles strongly adsorb DNA, allow for high loading efficiency, protect against degradation of the adsorbed DNA and enhance gene expression in muscle and local lymph nodes. Furthermore, DNA delivered using microparticles, as opposed to DNA delivered alone, is also able to recruit significant numbers of activated APC to the injection site following immunization. Thus, the use of such combinations provides a safe and effective approach for enhancing the immunogenicity of HCV E1E2 antigens.

Accordingly, in one embodiment, the invention is directed to a composition consisting essentially of a pharmaceutically acceptable excipient and a polynucleotide adsorbed to a cationic microparticle. The polynucleotide comprises a coding sequence that encodes a hepatitis C virus (HCV) immunogen operably linked to control elements that direct the transcription and translation of the coding sequence in vivo. The HCV immunogen is an immunogenic HCV E1E2 complex with a contiguous sequence of amino acids having at least 80% sequence identity to the contiguous sequence of amino acids depicted at positions 192-809 of FIGS. 2A-2C, with the proviso that the polynucleotide does not encode an HCV immunogen other than the HCV E1E2 complex.

In certain embodiments, the HCV E1E2 complex consists of the sequence of amino acids depicted at positions 192-809 of FIGS. 2A-2C.

In further embodiments, the cationic microparticle is formed from a polymer selected from the group consisting of a poly(α-hydroxy acid), a polyhydroxy butyric acid, a polycaprolactone, a polyorthoester, and a polyanhydride, such as a poly(α-hydroxy acid) selected from the group consisting of poly(L-lactide), poly(D,L-lactide) and poly(DL-lactide-co-glycolide).

In additional embodiments, the invention is directed to a composition consisting essentially of: (a) a pharmaceutically acceptable excipient; and (b) a polynucleotide adsorbed to a cationic microparticle formed from poly(D,L-lactide-co-glycolide). The polynucleotide comprises a coding sequence that encodes a hepatitis C virus (HCV) immunogen operably linked to control elements that direct the transcription and translation of the coding sequence in vivo, and the HCV immunogen is an HCV E1E2 complex consisting of the sequence of amino acids depicted at positions 192-809 of FIGS. 2A-2C, with the proviso that the polynucleotide does not encode an HCV immunogen other than the HCV E1E2 complex.

In yet further embodiments, the invention is directed to a method of stimulating an immune response in a vertebrate subject which comprises administering to the subject a therapeutically effective amount of a first composition consisting essentially of a pharmaceutically acceptable excipient and a polynucleotide adsorbed to a cationic microparticle. The polynucleotide comprises a coding sequence that encodes a hepatitis C virus (HCV) immunogen operably linked to control elements that direct the transcription and translation of the coding sequence in vivo. The HCV immunogen is an immunogenic HCV E1E2 complex with a contiguous sequence of amino acids having at least 80% sequence identity to the contiguous sequence of amino acids depicted at positions 192-809 of FIGS. 2A-2C, with the proviso that the polynucleotide does not encode an HCV immunogen other than the HCV E1E2 complex, wherein the HCV E1E2 complex is expressed in vivo to elicit an immune response.

In certain embodiments, the HCV E1E2 complex consists of the sequence of amino acids depicted at positions 192-809 of FIGS. 2A-2C.

In further embodiments, the cationic microparticle is formed from a polymer selected from the group consisting of a poly(α-hydroxy acid), a polyhydroxy butyric acid, a polycaprolactone, a polyorthoester, and a polyanhydride, such as a poly(α-hydroxy acid) selected from the group consisting of poly(L-lactide), poly(D,L-lactide) and poly(D,L-lactide-co-glycolide).

In additional embodiments, the method further comprises administering a therapeutically effective amount of a second composition to the subject, wherein the second composition comprises an immunogenic HCV polypeptide and a pharmaceutically acceptable excipient.

In certain embodiments the second composition is administered subsequent to the first composition. Additionally, the immunogenic HCV polypeptide in the second composition can be an immunogenic HCV E1E2 complex with a contiguous sequence of amino acids having at least 80% sequence identity to the contiguous sequence of amino acids depicted at positions 192-809 of FIGS. 2A-2C. In an additional embodiment, the HCV E1E2 complex consists of the sequence of amino acids depicted at positions 192-809 of FIGS. 2A-2C.

In a further embodiment, the second composition further comprises an adjuvant, such as a submicron oil-in-water emulsion capable of enhancing the immune response to the immunogenic HCV polypeptide. The submicron oil-in-water emulsion comprises (i) a metabolizable oil, wherein the oil is present in an amount of 1% to 12% of the total volume, and (ii) an emulsifying agent, wherein the emulsifying agent is present in an amount of 0.01% to 1% by weight (w/v) and comprises polyoxyethylene sorbitan mono-, di-, or triester and/or a sorbitan mono-, di-, or triester, wherein the oil and the emulsifying agent are present in the form of an oil-in-water emulsion having oil droplets substantially all of which are about 100 nm to less than 1 micron in diameter.

In certain embodiments, the submicron oil-in-water emulsion comprises 4-5% w/v squalene, 0.25-1.0% w/v polyoxyelthylenesorbitan monooleate, and/or 0.25-1.0% sorbitan trioleate, and optionally, N-acetylmuramyl-L-alanyl-D-isogluatminyl-L-alanine-2-(1′-2′-dipalmitoyl-sn-glycero-3-huydroxyphosphoryloxy)-ethylamine (MTP-PE).

In additional embodiments, the submicron oil-in-water emulsion consists essentially of about 5% by volume of squalene; and one or more emulsifying agents selected from the group consisting of polyoxyelthylenesorbitan monooleate and sorbitan trioleate, wherein the total amount of emulsifying agent(s) present is about 1% by weight (w/v).

In further embodiments, the one or more emulsifying agents are polyoxyelthylenesorbitan monooleate and sorbitan trioleate and the total amount of polyoxyelthylenesorbitan monooleate and sorbitan trioleate present is about 1% by weight (w/v).

In yet additional embodiments, the second composition further comprises a CpG oligonucleotide.

In another embodiment, the invention is directed to a method of stimulating an immune response in a vertebrate subject which comprises:

(a) administering to the subject a therapeutically effective amount of a first composition consisting essentially of a polynucleotide adsorbed to a cationic microparticle formed from poly(D,L-lactide-co-glycolide), wherein the polynucleotide comprises a coding sequence that encodes a hepatitis C virus (HCV) immunogen operably linked to control elements that direct the transcription and translation of the coding sequence in vivo, and further wherein the HCV immunogen is an HCV E1E2 complex consisting of the sequence of amino acids depicted at positions 192-809 of FIGS. 2A-2C, with the proviso that the polynucleotide does not encode an HCV immunogen other than the HCV E1E2 complex, and wherein the HCV E1E2 complex is expressed in vivo; and

(b) administering a therapeutically effective amount of a second composition to the subject, wherein the second composition comprises (i) an immunogenic HCV E1E2 complex consisting of the sequence of amino acids depicted at positions 192-809 of FIGS. 2A-2C, (ii) an adjuvant, and (iii) a pharmaceutically acceptable excipient, to elicit an immune response in the subject.

In certain embodiments, the adjuvant is a submicron oil-in-water emulsion capable of enhancing the immune response to the immunogenic HCV E1E2 complex in the second composition. The submicron oil-in-water emulsion comprises (i) a metabolizable oil, wherein the oil is present in an amount of 1% to 12% of the total volume, and (ii) an emulsifying agent, wherein the emulsifying agent is present in an amount of 0.01% to 1% by weight (w/v) and comprises polyoxyethylene sorbitan mono-, di-, or triester and/or a sorbitan mono-, di-, or triester, wherein the oil and the emulsifying agent are present in the form of an oil-in-water emulsion having oil droplets substantially all of which are about 100 nm to less than 1 micron in diameter.

In additional embodiments, the submicron oil-in-water emulsion comprises 4-5% w/v squalene, 0.25-1.0% w/v polyoxyelthylenesorbitan monooleate, and/or 0.25-1.0% sorbitan trioleate, and optionally, N-acetylmuramyl-L-alanyl-D-isogluatminyl-L-alanine-2-(1′-2′-dipalmitoyl-sn-glycero-3-huydroxyphosphoryloxy)-ethylamine (MTP-PE).

In further embodiments, the submicron oil-in-water emulsion consists essentially of about 5% by volume of squalene; and one or more. emulsifying agents selected from the group consisting of polyoxyelthylenesorbitan monooleate and sorbitan trioleate, wherein the total amount of emulsifying agent(s) present is about 1% by weight (w/v).

In additional embodiments, the one or more emulsifying agents are polyoxyelthylenesorbitan monooleate and sorbitan trioleate and the total amount of polyoxyelthylenesorbitan monooleate and sorbitan trioleate present is about 1% by weight (w/v).

In certain embodiments, the second composition further comprises a CpG oligonucleotide.

In yet a further embodiment, the invention is directed to a method of making a composition comprising combining a pharmaceutically acceptable excipient with a polynucleotide adsorbed to a cationic microparticle. The polynucleotide comprises a coding sequence that encodes a hepatitis C virus (HCV) immunogen operably linked to control elements that direct the transcription and translation of the coding sequence in vivo. The HCV immunogen is an immunogenic HCV E1E2 complex with a contiguous sequence of amino acids having at least 80% sequence identity to the contiguous sequence of amino acids depicted at positions 192-809 of FIGS. 2A-2C, with the proviso that said polynucleotide does not encode an HCV immunogen other than the HCV E1E2 complex.

These and other embodiments of the subject invention will readily occur to those of skill in the art in view of the disclosure herein.

### BRIEF DESCRIPTION OF THE FIGURES

FIG. 1 is a diagrammatic representation of the HCV genome, depicting the various regions of the HCV polyprotein.

FIGS. 2A-2C (SEQ ID NOS:1 and 2) show the nucleotide and corresponding amino acid sequence for the HCV-1 E1/E2/p7 region. The numbers shown in the figure are relative to the full-length HCV-1 polyprotein. The E1, E2 and p7 regions are shown.

FIG. 3 shows serum IgG titers following immunization of mice at 0 and 4 weeks with E1E2 plasmid DNA alone or PLG/CTAB/E1E2₈₀₉DNA (indicated as PLG/DNA in the figures) at 10 μg and 100 μg (N=10, +/−SEM).

FIG. 4 shows serum IgG titers following immunization of mice at 0 and 4 weeks with E1E2₈₀₉ plasmid DNA at 10 μg, PLG/CTAB/E1E2₈₀₉DNA at 1 μg and 10 μg, or E1E2 E1E2₈₀₉ recombinant protein in MF59 adjuvant at 2 μg (N =10, +/−SEM).

FIG. 5 shows serum IgG titers following immunization of mice at 0, 4 and 8 weeks with E1E809 plasmid DNA or PLG/CTAB/E1E2₈₀₉DNA at 10 μg, or E1E2₈₀₉ recombinant protein in MF59 adjuvant at 5 μg. In addition, 2 groups of mice were immunized twice with E1E2₈₀₉ plasmid DNA or PLG/CTAB/ E1E2₈₀₉DNA 10 μg at 0 and 4 weeks, and boosted with 5 μg E1E2₈₀₉ recombinant protein in MF59 at 8 weeks (N =10, +/−SEM). D=E1E2₈₀₉DNA 10 μg; P=5 μg E1E2 protein in MF59.

### DETAILED DESCRIPTION OF THE INVENTION

The practice of the present invention will employ, unless otherwise indicated, conventional methods of chemistry, biochemistry, recombinant DNA techniques and immunology, within the skill of the art. Such techniques are explained fully in the literature. See, e.g., Fundamental Virology, 2nd Edition, vol. I & II (B. N. Fields and D. M. Knipe, eds.); Handbook of Experimental Immunology, Vols. I-IV (D. M. Weir and C. C. Blackwell eds., Blackwell Scientific Publications); T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| Tyrosine: Tyr (Y) | Valine: Val (V) |

### 1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an E1E2 polypeptide” includes a mixture of two or more such polypeptides, and the like.

The terms “polypeptide” and “protein” refer to a polymer of amino acid residues and are not limited to a minimum length of the product Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

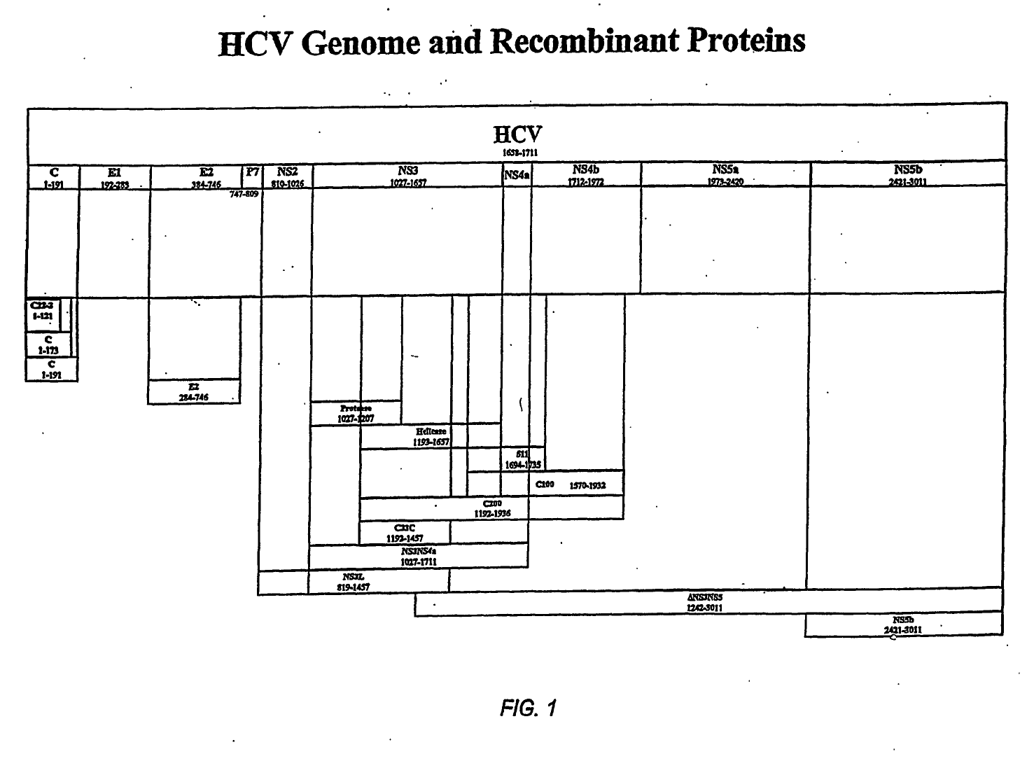

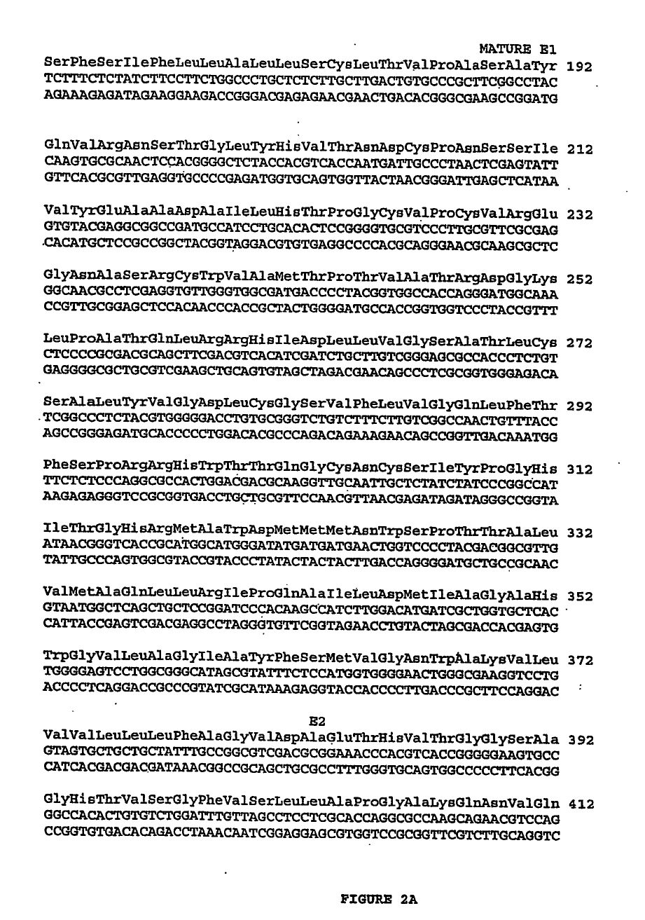

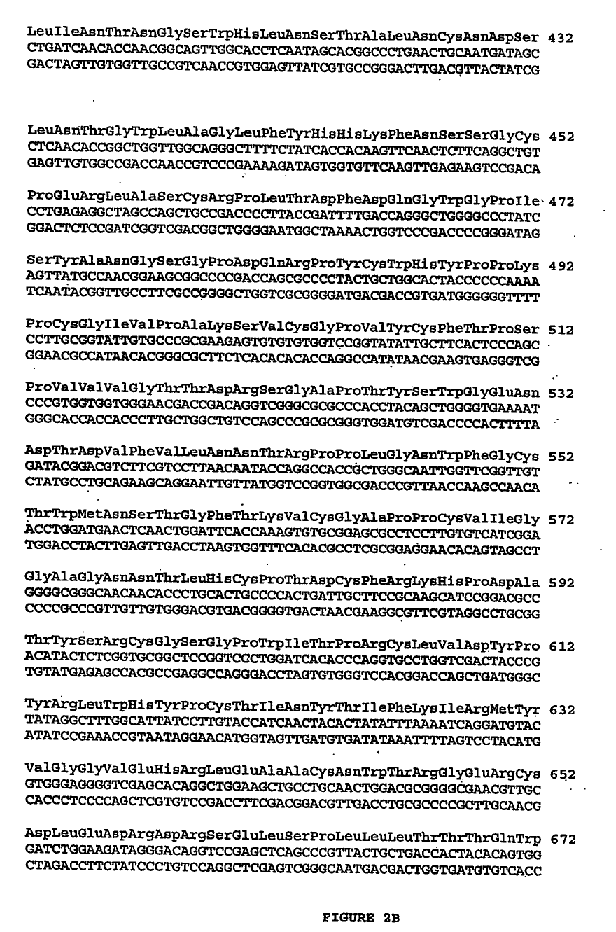

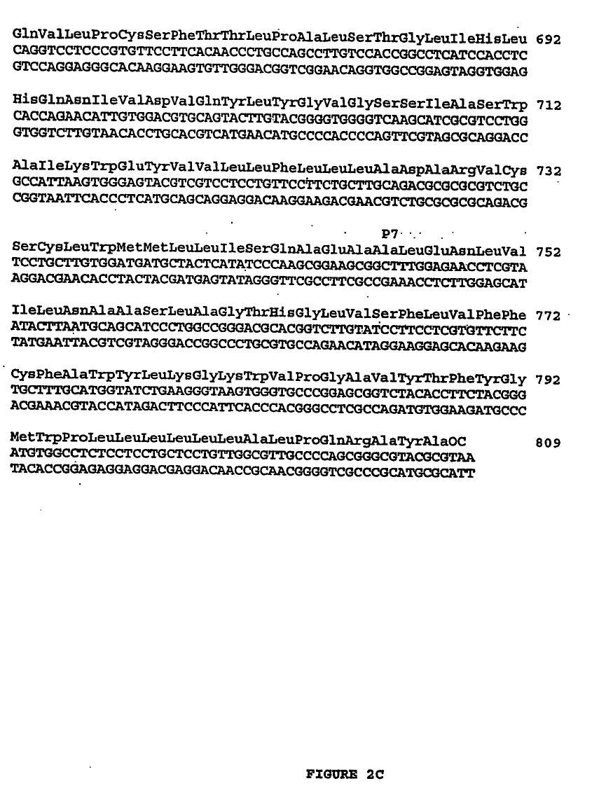

By an “E1 polypeptide” is meant a molecule derived from an HCV E1 region. The mature E1 region of HCV-1 begins at approximately amino acid 192 of the polyprotein and continues to approximately amino acid 383, numbered relative to the full-length HCV-1 polyprotein. (See, FIGS. 1 and 2A-2C. Amino acids 192-383 of FIGS. 2A-2C correspond to amino acid positions 20-211 of SEQ ID NO:2.) Amino acids at around 173 through approximately 191 (amino acids 1-19 of SEQ ID NO:2) serve as a signal sequence for E1. Thus, by an “E1 polypeptide” is meant either a precursor E1 protein, including the signal sequence, or a mature E1 polypeptide which lacks this sequence, or even an E1 polypeptide with a heterologous signal sequence. The E1 polypeptide includes a C-terminal membrane anchor sequence which occurs at approximately amino acid positions 360-383 (see, International Publication No. WO 96/04301, published Feb. 15, 1996). An E1 polypeptide, as defined herein, may or may not include the C-terminal anchor sequence or portions thereof.

By an “E2 polypeptide” is meant a molecule derived from an HCV E2 region. The mature E2 region of HCV-1 begins at approximately amino acid 383-385, numbered relative to the full-length HCV-1 polyprotein. (See, FIGS. 1 and 2A-2C. Amino acids 383-385 of FIGS. 2A-2C correspond to amino acid positions 211-213 of SEQ ID NO:2.) A signal peptide begins at approximately amino acid 364 of the polyprotein. Thus, by an “E2 polypeptide” is meant either a precursor E2 protein, including the signal sequence, or a mature E2 polypeptide which lacks this sequence, or even an E2 polypeptide with a heterologous signal sequence. The E2 polypeptide includes a C-terminal membrane anchor sequence which occurs at approximately amino acid positions 715-730 and may extend as far as approximately amino acid residue 746 (see, Lin et al., J. Virol. (1994) 68:5063-5073). An E2 polypeptide, as defined herein, may or may not include the C-terminal anchor sequence or portions thereof. Moreover, an E2 polypeptide may also include all or a portion of the p7 region which occurs immediately adjacent to the C-terminus of E2. As shown in FIGS. 1 and 2A-2C, the p7 region is found at positions 747-809, numbered relative to the full-length HCV-1 polyprotein (amino acid positions 575-637 of SEQ ID NO:2). Additionally, it is known that multiple species of HCV E2 exist (Spaete et al., Virol. (1992)188:819-830; Selby et al., J. Virol. (1996) 70:5177-5182; Grakoui et al., J. Virol. (1993) 67:1385-1395; Tomei et al., J. Virol. (1993)67:40174026). Accordingly, for purposes of the present invention, the term “E2” encompasses any of these species of E2 including, without limitation, species that have deletions of 1-20 or more of the amino acids from the N-terminus of the E2, such as, e.g, deletions of 1, 2, 3, 4, 5 . . . 10 . . . 15, 16, 17, 18, 19 . . . etc. amino acids. Such E2 species include those beginning at amino acid 387, amino acid 402, amino acid 403, etc.

Representative E1 and E2 regions from HCV-1 are shown in FIGS. 2A-2C and SEQ ID NO:2. For purposes of the present invention, the E1 and E2 regions are defined with respect to the amino acid number of the polyprotein encoded by the genome of HCV-1, with the initiator methionine being designated position 1. See, e.g., Choo et al., Proc. Natl. Acad. Sci. USA (1991) 88:2451-2455. However, it should be noted that the term an “E1 polypeptide” or an “E2 polypeptide” as used herein is not limited to the HCV-1 sequence. In this regard, the corresponding E1 or E2 regions in other HCV isolates can be readily determined by aligning sequences from the isolates in a manner that brings the sequences into maximum alignment This can be performed with any of a number of computer software packages, such as ALIGN 1.0, available from the University of Virginia, Department of Biochemistry (Attn: Dr. William R Pearson). See, Pearson et al., Proc. Natl. Acad. Sci USA (1988) 85:2444-2448.

Furthermore, an “E1 polypeptide” or an “E2 polypeptide” as defined herein is not limited to a polypeptide having the exact sequence depicted in the Figures. Indeed, the HCV genome is in a state of constant flux in vivo and contains several variable domains which exhibit relatively high degrees of variability between isolates. A number of conserved and variable regions are known between these strains and, in general, the amino acid sequences of epitopes derived from these regions will have a high degree of sequence homology, e.g., amino acid sequence homology of more than 30%, preferably more than 40%, more than 60%, and even more than 80-90% homology, when the two sequences are aligned. It is readily apparent that the terms encompass E1 and E2 polypeptides from any of the various HCV strains and isolates including isolates having any of the 6 genotypes of HCV described in Simmonds et al., J. Gen. Virol. (1993) 74:2391-2399 (e.g., strains 1, 2, 3,4 etc.), as well as newly identified isolates, and subtypes of these isolates, such as HCV1a, HCV1b etc.

Thus, for example, the term “E1” or “E2” polypeptide refers to native E1 or E2 sequences from any of the various HCV strains, as well as analogs, muteins and immunogenic fragments, as defined further below. The complete genotypes of many of these strains are known. See, e.g., U.S. Pat. No. 6,150,087 and GenBank Accession Nos. AJ238800 and AJ238799.

Additionally, the terms “E1 polypeptide” and “E2 polypeptide” encompass proteins which include modifications to the native sequence, such as internal deletions, additions and substitutions (generally conservative in nature), such as proteins substantially homologous to the parent sequence. These modifications may be deliberate, as through site-directed mutagenesis, or may be accidental, such as through naturally occurring mutational events. All of these modifications are encompassed in the present invention so long as the modified E1 and E2 polypeptides function for their intended purpose. Thus, for example, if the E1 and/or E2 polypeptides are to be used in vaccine compositions, the modifications must be such that immunological activity (i.e., the ability to elicit a humoral or cellular immune response to the polypeptide) is not lost By “E1E2” complex is meant a protein containing at least one E1 polypeptide and at least one E2 polypeptide, as described above. Such a complex may also include all or a portion of the p7 region which occurs immediately adjacent to the C-terminus of E2. As shown in FIGS. 1 and 2A-2C, the p7 region is found at positions 747-809, numbered relative to the full-length HCV-1 polyprotein (amino acid positions 575-637 of SEQ ID NO:2). A representative E1E2 complex which includes the p7 protein is termed “E1E2₈₀₉” herein.

The mode of association of E1 and E2 in an E1E2 complex is immaterial. The E1 and E2 polypeptides may be associated through non-covalent interactions such as through electrostatic forces, or by covalent bonds. For example, the E1E2 polypeptides of the present invention may be in the form of a fusion protein which includes an immunogenic E1 polypeptide and an immunogenic E2 polypeptide, as defined above. The fusion may be expressed from a polynucleotide encoding an E1E2 chimera. Alternatively, E1E2 complexes may form spontaneously simply by mixing E1 and E2 proteins which have been produced individually. Similarly, when co-expressed and secreted into media, the E1 and E2 proteins can form a complex spontaneously. Thus, the term encompasses E1E2 complexes (also called aggregates) that spontaneously form upon purification of E1 and/or E2. Such aggregates may include one or more E1 monomers in association with one or more E2 monomers. The number of E1 and E2 monomers present need not be equal so long as at least one E1 monomer and one E2 monomer are present Detection of the presence of an E1E2 complex is readily determined using standard protein detection techniques such as polyacrylamide gel electrophoresis and immunological techniques such as immunoprecipitation.

The terms “analog” and “mutein” refer to biologically active derivatives of the reference molecule, such as E1E2₈₀₉, or fragments of such derivatives, that retain desired activity, such as immunoreactivity in assays described herein. In general, the term “analog” refers to compounds having a native polypeptide sequence and structure with one or more amino acid additions, substitutions (generally conservative in nature) and/or deletions, relative to the native molecule, so long as the modifications do not destroy immunogenic activity. The term “mutein” refers to peptides having one or more peptide mimics (“peptoids”), such as those described in International Publication No. WO 91/04282. Preferably, the analog or mutein has at least the same immunoreactivity as the native molecule. Methods for making polypeptide analogs and muteins are known in the art and are described further below.

Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest, such as an E1E2 polypeptide, may include up to about 5-10 conservative or non-conservative amino acid substitutions, or even up to about 15-25 or 50 conservative or non-conservative amino acid substitutions, or any integer between 5-50, so long as the desired function of the molecule remains intact. One of skill in the art can readily determine regions of the molecule of interest that can tolerate change by reference to Hopp/Woods and Kyte-Doolittle plots, well known in the art

By “fragment” is intended a polypeptide consisting of only a part of the intact full-length polypeptide sequence and structure. The fragment can include a C-terminal deletion an N-terminal deletion, and/or an internal deletion of the native polypeptide. An “immunogenic fragment” of a particular HCV protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains the ability to elicit an immunological response as defined herein. For a description of known immunogenic fragments of HCV E1 and E2, see, e.g., Chien et al., International Publication No. WO 93/00365.

The term “epitope” as used herein refers to a sequence of at least about 3 to 5, preferably about 5 to 10 or 15, and not more than about 500 amino acids (or any integer therebetween), which define a sequence that by itself or as part of a larger sequence, elicits an immunological response in the subject to which it is administered. Often, an epitope will bind to an antibody generated in response to such sequence. There is no critical upper limit to the length of the fragment, which may comprise nearly the full-length of the protein sequence, or even a fusion protein comprising two or more epitopes from the HCV polyprotein. An epitope for use in the subject invention is not limited to a polypeptide having the exact sequence of the portion of the parent protein from which it is derived. Indeed, viral genomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

Regions of a given polypeptide that include an epitope can be identified using any number of epitope mapping techniques, well known in the art See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1985) Proc. Natl. Acad. Sci. USA 82:178-182; Geysen et al. (1986) Molec. Immunol. 23:709-715. Using such techniques, a number of epitopes of HCV have been identified. See, e.g., Chien et al., Viral Hepatitis and Liver Disease (1994) pp. 320-324, and further below. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Nat. Acad. Sci USA (1981) 78:3824-3828 for determining antigenicity profiles, and the Kyte-Doolittle technique, Kyte et al., J. Mol. Biol. (1982) 157:105-132 for hydropathy plots.

As used herein, the term “conformational epitope” refers to a portion of a full-length protein, or an analog or mutein thereof having structural features native to the amino acid sequence encoding the epitope within the full-length natural protein. Native structural features include, but are not limited to, glycosylation and three dimensional structure. The length of the epitope defining sequence can be subject to wide variations as these epitopes are believed to be formed by the three-dimensional shape of the antigen (e.g., folding). Thus, amino acids defining the epitope can be relatively few in number, but widely dispersed along the length of the molecule (or even on different molecules in the case of dimers, etc.), being brought into correct epitope conformation via folding. The portions of the antigen between the residues defining the epitope may not be critical to the conformational structure of the epitope. For example, deletion or substitution of these intervening sequences may not affect the conformational epitope provided sequences critical to epitope conformation are maintained (e.g., cysteines involved in disulfide bonding, glycosylation sites, etc.).

Conformational epitopes are readily identified using methods discussed above. Moreover, the presence or absence of a conformational epitope in a given polypeptide can be readily determined through screening the antigen of interest with an antibody (polyclonal serum or monoclonal to the conformational epitope) and comparing its reactivity to that of a denatured version of the antigen which retains only linear epitopes (if any). In such screening using polyclonal antibodies, it may be advantageous to absorb the polyclonal serum first with the denatured antigen and see if it retains antibodies to the antigen of interest Conformational epitopes derived from the E1 and E2 regions are described in, e.g., International Publication No. WO 94/01778.

An “immunological response” to an HCV antigen or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject Such assays are well known in the art See, e.g., Erickson et al., J. Immunol. (1993) 151:4189-4199; Doe et al., Eur. J. Immunol. (1994) 24:2369-2376.

Thus, an immunological response as used herein may be one which stimulates the production of CTLs, and/or the production or activation of helper T- cells. The antigen of interest may also elicit an antibody-mediated immune response, including, or example, neutralization of binding (NOB) antibodies. The presence of an NOB antibody response is readily determined by the techniques described in, e.g., Rosa et al., Proc. Natl. Acad. Sci. USA (1996) 93:1759. Hence, an immunological response may include one or more of the following effects: the production of antibodies by B-cells; and/or the activation of suppressor T-cells and/or γT-cells directed specifically to an antigen or antigens present in the composition or vaccine of interest. These responses may serve to neutralize infectivity, and/or mediate antibody-complement, or antibody dependent cell cytotoxicity (ADCC) to provide protection or alleviation of symptoms to an immunized host Such responses can be determined using standard immunoassays and neutralization assays, well known in the art.

A component of an HCV E1E2 DNA composition, such as a cationic microparticle, enhances the immune response to the HCV E1E2 polypeptide produced by the DNA in the composition when the composition possesses a greater capacity to elicit an immune response than the immune response elicited by an equivalent amount of the E1E2 DNA delivered without the cationic microparticle. Such enhanced immunogenicity can be determined by administering the E1E2 DNA with and without the additional components, and comparing antibody titers or cellular immune response produced by the two using standard assays such as radioimmunoassay, ELISAs, lymphoproliferation assays, and the like, well known in the art.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

By “equivalent antigenic determinant” is meant an antigenic determinant from different sub-species or strains of HCV, such as from strains 1, 2, 3, etc., of HCV which antigenic determinants are not necessarily identical due to sequence variation, but which occur in equivalent positions in the HCV sequence in question. In general the amino acid sequences of equivalent antigenic determinants will have a high degree of sequence homology, e.g., ammo acid sequence homology of more than 30%, usually more than 40%, such as more than 60%, and even more than 80-90% homology, when the two sequences are aligned.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Wash. DC, which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

By the term “degenerate variant” is intended a polynucleotide containing changes in the nucleic acid sequence thereof, that encodes a polypeptide having the same amino acid sequence as the polypeptide encoded by the polynucleotide from which the degenerate variant is derived. Thus, a degenerate variant of E1E2₈₀₉ DNA is a molecule with one or more base differences in the DNA sequence from which the molecule is derived but that encodes the same E1E2₈₀₉ amino acid sequence.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

A “nucleic acid” molecule or “polynucleotide” can include both double- and single-stranded sequences and refers to, but is not limited to, cDNA from viral, procaryotic or eucaryotic mRNA, genomic DNA sequences from viral (e.g. DNA viruses and retroviruses) or procaryotic DNA, and synthetic DNA sequences. The term also captures sequences that include any of the known base analogs of DNA and RNA.

An “HCV polynucleotide” is a polynucleotide that encodes an HCV polypeptide, as defined above.

“Operably linked” refers to an arrangement of elements wherein the components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can transcribed introns, and the promoter sequence can still be considered “operably linked” to the coding sequence.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

A “control element” refers to a polynucleotide sequence which aids in the expression of a coding sequence to which it is linked. The term includes promoters, transcription termination sequences, upstream regulatory domains, polyadenylation signals, untranslated regions, including 5′-UTRs and 3′-UTRs and when appropriate, leader sequences and enhancers, which collectively provide for the transcription and translation of a coding sequence in a host cell.

A “promoter” as used herein is a DNA regulatory region capable of binding RNA polymerase in a host cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

A control sequence “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest The expression cassette includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

“Transformation,” as used herein, refers to the insertion of an exogenous polynucleotide into a host cell, irrespective of the method used for insertion: for example, transformation by direct uptake, transfection, infection, and the like. For particular methods of transfection, see further below. The exogenous polynucleotide may be maintained as a nonintegrated vector, for example, an episome, or alternatively, may be integrated into the host genome.

By “nucleic acid immunization” is meant the introduction of a nucleic acid molecule encoding one or more selected immunogens, such as E1E2, into a host cell, for the in vivo expression of the immunogen. The nucleic acid molecule can be introduced directly into a recipient subject, such as by injection, inhalation, oral intranasal and mucosal administration, or the like, or can be introduced ex vivo, into cells which have been removed from the host. In the latter case, the transformed cells are reintroduced into the subject where an immune response can be mounted against the immunogen encoded by the nucleic acid molecule.

The terms “effective amount” or “pharmaceutically effective amount” of an immunogenic composition, as provided herein, refer to a nontoxic but sufficient amount of the composition to provide the desired response, such as an immunological response, and optionally, a corresponding therapeutic effect. The exact amount required will vary from subject to subject, depending on the species, age, and general condition of the subject, the severity of the condition being treated, and the particular macromolecule of interest, mode of administration, and the like. An appropriate “effective” amount in any individual case may be determined by one of ordinary skill in the art using routine experimentation.

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species, since the immune systems of all of these vertebrates operate similarly.

The term “treatment” as used herein refers to either (1) the prevention of infection or reinfection (prophylaxis), or (2) the reduction or elimination of symptoms of the disease of interest (therapy).

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Central to the present invention is the discovery that plasmid DNA encoding HCV E1E2 envelope protein adsorbed onto cationic microparticles induces significantly enhanced antibody responses as compared to the use of non-adsorbed plasmid E1E2 DNA. Moreover, the adsorbed DNA induces detectable responses at a dose an order of magnitude lower than the dose required to produce detectable antibodies with the non-adsorbed DNA. Additionally, the antibody response induced by the adsorbed DNA is comparable to the response achieved by administration of the E1E2 protein while delivery of the non-adsorbed E1E2 DNA barely induces detectable responses. E1E2 DNA adsorbed to cationic microparticles is more effective at priming for potent responses following booster immunizations with recombinant protein than with the plasmid DNA alone. Moreover, the examples below evidence the ability of adsorbed E1E2 DNA to produce a cellular immune response.

Thus, as described in more detail below, subjects are initially administered DNA encoding E1E2₈₀₉ complexes adsorbed to cationic microparticles. Subjects can subsequently be boosted with DNA compositions comprising DNA encoding E1E2 complexes and/or protein compositions comprising E1E2 protein complexes. The E1E2 complexes used for boosting can be either E1E2₈₀₉, or can be other E1E2 proteins, as described further below, so long as an immune response is generated. Additionally, the compositions above can be used alone, or in combination with other compositions, such as compositions comprising other HCV proteins, compositions comprising DNA encoding other HCV proteins, as well as compositions comprising ancillary substances. If used in combination with other compositions, such compositions can be administered prior to, concurrent with, or subsequent to the E1E2 compositions.

In order to further an understanding of the invention, a more detailed discussion is provided below regarding E1E2 DNA and protein compositions, cationic microparticles, and additional compositions for use in the subject methods.

E1E2 Polypeptides and Polynucleotides

E1E2 complexes comprise E1 and E2 polypeptides, associated either through non-covalent or covalent interactions. As explained above, the HCV E1 polypeptide is a glycoprotein and extends from approximately amino acid 192 to amino acid 383 (numbered relative to the polyprotein of HCV-1). See, Choo et al., Proc. Natl. Acad. Sci. USA (1991) 88:2451-2455. Amino acids at around 173 through approximately 191 represent a signal sequence for E1. An HCV E2 polypeptide is also a glycoprotein and extends from approximately amino acid 383 or 384 to amino acid 746. A signal peptide for E2 begins at approximately amino acid 364 of the polyprotein. Thus, the term “full-length” E1 or “not truncated” E1 as used herein refers to polypeptides that include, at least, amino acids 192-383 of an HCV polyprotein (numbered relative 'to HCV-1). With respect to E2, the term “full-length” or “not truncated” as used herein refers to polypeptides that include, at least, amino acids 383 or 384 to amino acid 746 of an HCV polyprotein (numbered relative to HCV-1). As will be evident from this disclosure, E2 polypeptides for use with the present invention may include additional amino acids from the p7 region, such as amino acids 747-809.

E2 exists as multiple species (Spaete et al., Virol. (1992) 188:819-830; Selby et al., J. Virol. (1996) 70:5177-5182; Grakoui et al., J. Virol. (1993) 67:1385-1395; Tomei et al., J. Virol. (1993) 67:4017-4026) and clipping and proteolysis may occur at the N- and C-termini of the E1 and E2 polypeptides. Thus, an E2 polypeptide for use herein may comprise at least amino acids 405-661, e.g., 400, 401, 402 . . . to 661, such as 383 or 384-661, 383 or 384-715, 383 or 384-746, 383 or 384-749 or 383 or 384-809, or 383 or 384 to any C-terminus between 661-809, of an HCV polyprotein, numbered relative to the full-length HCV-1 polyprotein. Similarly, preferable E1polypeptides for use herein can comprise amino acids 192-326, 192-330, 192-333, 192-360, 192-363, 192-383, or 192 to any C-terminus between 326-383, of an HCV polyprotein.

The E1E2 complexes may also be made up of immunogenic fragments of E1and E2 which comprise epitopes. For example, fragments of E1 polypeptides can comprise from about 5 to nearly the full-length of the molecule, such as 6, 10, 25, 50, 75, 100, 125, 150, 175, 185 or more amino acids of an E1 polypeptide, or any integer between the stated numbers. Similarly, fragments of E2 polypeptides can comprise 6, 10, 25, 50, 75, 100, 150, 200, 250, 300, or 350 amino acids of an E2 polypeptide, or any integer between the stated numbers. The E1 and E2 polypeptides may be from the same or different HCV strains.

For example, epitopes derived from, e.g., the hypervariable region of E2, such as a region spanning amino acids 384-410 or 390-410, can be included in the E2 polypeptide. A particularly effective E2 epitope to incorporate into the E2 sequence is one which includes a consensus sequence derived from this region, such as the consensus sequence Gly-Ser-Ala-Ala-Arg-Thr-Thr-Ser-Gly-Phe-Val-Ser-Leu-Phe-Ala-Pro-Gly-Ala-Lys-Gln-Asn, which represents a consensus sequence for amino acids 390-410 of the HCV type 1 genome. Additional epitopes of E1 and E2 are known and described in, e.g., Chien et al., International Publication No. WO 93/00365.

Moreover, the E1 and E2 polypeptides of the complex may lack all or a portion of the membrane spanning domain. The membrane anchor sequence functions to associate the polypeptide to the endoplasmic reticulum. Normally, such polypeptides are capable of secretion into growth medium in which an organism expressing the protein is cultured. However, as described in International Publication No. WO 98/50556, such polypeptides may also be recovered intracellularly. Secretion into growth medium is readily determined using a number of detection techniques, including, e.g., polyacrylamide gel electrophoresis and the like, and immunological techniques such as immunoprecipitation assays as described in, e.g., International Publication No. WO 96/04301, published Feb. 15, 1996. With E1, generally polypeptides terminating with about amino acid position 370 and higher (based on the numbering of HCV-1 E1) will be retained by the ER and hence not secreted into growth media With E2, polypeptides terminating with about amino acid position 731 and higher (also based on the numbering of the HCV-1 E2 sequence) will be retained by the ER and not secreted. (See, e.g., International Publication No. WO 96/04301, published Feb. 15, 1996). It should be noted that these amino acid positions are not absolute and may vary to some degree. Thus, the present invention contemplates the use of E1 and E2 polypeptides which retain the transmembrane binding domain, as well as polypeptides which lack all or a portion of the transmembrane binding domain, including E1 polypeptides terminating at about amino acids 369 and lower, and E2 polypeptides, terminating at about amino acids 730 and lower, are intended to be captured by the present invention. Furthermore, the C-terminal truncation can extend beyond the transmembrane spanning domain towards the N-terminus. Thus, for example, E1 truncations occurring at positions lower than, e.g., 360 and E2 truncations occurring at positions lower than, e.g., 715, are also encompassed by the present invention. All that is necessary is that the truncated E1 and E2 polypeptides remain functional for their intended purpose. However, particularly preferred truncated E1 constructs are those that do not extend beyond about amino acid 300. Most preferred are those terminating at position 360. Preferred truncated E2 constructs are those with C-terminal truncations that do not extend beyond about amino acid position 715. Particularly preferred E2 truncations are those molecules truncated after any of amino acids 715-730, such as 725. If truncated molecules are used, it is preferable to use E1 and E2 molecules that are both truncated.

The E1 and E2 polypeptides and complexes thereof may also be present as asialoglycoproteins. Such asialoglycoproteins are produced by methods known in the art, such as by using cells in which terminal glycosylation is blocked. When these proteins are expressed in such cells and isolated by GNA lectin affinity chromatography, the E1 and E2 proteins aggregate spontaneously. Detailed methods for producing these E1E2 aggregates are described in, e.g., U.S. Pat. No. 6,074,852.

Moreover, the E1E2 complexes may comprise a heterogeneous mixture of molecules, due to clipping and proteolytic cleavage, as described above. Thus, a composition including E1E2 complexes may include multiple species of E1E2, such as E1E2 terminating at amino acid 746 (E1E2746), E1E2 terminating at amino acid 809 (E1E2₈₀₉), or any of the other various E1 and E2 molecules described above, such as E2 molecules with N-terminal truncations of from 1-20 amino acids, such as E2 species beginning at amino acid 387, amino acid 402, amino acid 403, etc.

It should be noted that for convenience, the E1 and E2 regions are generally defined with respect to the amino acid number relative to the polyprotein encoded by the genome of HCV-1a, as described in Choo et al. (1991) Proc Natl Acad Sci USA 88:2451, with the initiator methionine being designated position 1. However, the polypeptides for use with the present invention are not limited to those derived from the HCV-1a sequence. Any strain or isolate of HCV can serve as the basis for providing immunogenic sequences for use with the invention. In this regard, the corresponding regions in another HCV isolate can be readily determined by aligning sequences from the two isolates in a manner that brings the sequences into maximum alignment.

Various strains and isolates of HCV are known in the art, which differ from one another by changes in nucleotide and amino acid sequence. For example, isolate HCV J1.1 is described in Kubo et al. (1989) Japan. Nucl. Acids Res. 17:10367-10372; Takeuchi et al. (1990) Gene 91:287-291; Takeuchi et al. (1990) J. Gen. Virol. 71:3027-3033; and Takeuchi et al. (1990) Nucl. Acids Res. 18:4626. The complete coding sequences of two independent isolates, HCV-J and BK, are described by Kato et al., (1990) Proc. Natl. Acad. Sci. USA 87:9524-9528 and Takamizawa et al., (1991) J. Virol. 65:1105-1113, respectively. HCV-1 isolates are described by Choo et al. (1990) Brit. Med. Bull. 46:423-441; Choo et al. (1991) Proc. Natl. Acad. Sci. USA 88:2451-2455 and Han et al. (1991) Proc. Natl. Acad. Sci. USA 88:1711-1715. HCV isolates HC-J1 and HC-J4 are described in Okamoto et al. (1991) Japan J. Exp. Med. 60:167-177. HCV isolates HCT 18, HCT 23, Th, HCT 27, EC1 and EC10 are described in Weiner et al. (1991) Virol. 180:842-848. HCV isolates Pt-1, HCV-K1 and HCV-K2 are described in Enomoto et al. (1990) Biochem. Biophys. Res. Commun. 170:1021-1025. HCV isolates A, C, D & E are described in Tsukiyama-Kohara et al. (1991) Virus Genes 5:243-254. HCV E1E2 polynucleotides and polypeptides for use in the compositions and methods of the invention can be obtained from any of the above cited strains of HCV or from newly discovered isolates isolated from tissues or fluids of infected patients.

If delivery of E1E2 complexes as proteins is desired (e.g., for boosting an immune response) such E1E2 complexes are readily produced recombinantly, either as fusion proteins or by e.g., cotransfecting host cells with constructs encoding for the E1 and E2 polypeptides of interest Cotransfection can be accomplished either in trans or cis, i.e., by using separate vectors or by using a single vector which bears both of the E1 and E2 genes. If done using a single vector, both genes can be driven by a single set of control elements or, alternatively, the genes can be present on the vector in individual expression cassettes, driven by individual control elements. Following expression, the E1 and E2 proteins will spontaneously associate. Alternatively, the complexes can be formed by mixing the individual proteins together which have been produced separately, either in purified or semi-purified form, or even by mixing culture media in which host cells expressing the proteins, have been cultured, if the proteins are secreted. Finally, the E1E2 complexes of the present invention may be expressed as a fusion protein wherein the desired portion of E1 is fused to the desired portion of E2.

Methods for producing E1E2 complexes from full-length, truncated E1 and E2 proteins which are secreted into media, as well as intracellularly produced truncated proteins, are known in the art For example, such complexes may be produced recombinantly, as described in U.S. Pat. No. 6,121,020; Ralston et al., J. Virol. (1993) 67:6753-6761, Grakoui et al., J Virol. (1993) 67:1385-1395; and Lanford et al., Virology (1993) 197:225-235.

Thus, polynucleotides encoding HCV E1 and E2 polypeptides for use with the present invention can be made using standard techniques of molecular biology. For example, polynucleotide sequences coding for the above-described molecules can be obtained using recombinant methods, such as by screening cDNA and genomic libraries from cells expressing the gene, or by deriving the gene from a vector known to include the same. Furthermore, the desired gene can be isolated directly from viral nucleic acid molecules, using techniques described in the art, such as in Houghton et al., U.S. Pat. No. 5,350,671. The gene of interest can also be produced synthetically, rather than cloned. The molecules can be designed with appropriate codons for the particular sequence. The complete sequence is then assembled from overlapping oligonucleotides prepared by standard methods and assembled into a complete coding sequence. See, e.g., Edge (1981) Nature 292:756; Nambair et al. (1984) Science 223:1299; and Jay et al. (1984) J. Biol. Chem. 259:6311.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. See, e.g., Sambrook, supra. In particular, one method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al. (1991) Proc. Natl. Acad. Sci. USA 88:4084-4088. Additionally, oligonucleotide directed synthesis (Jones et al. (1986) Nature 54:75-82), oligonucleotide directed mutagenesis of preexisting nucleotide regions (Riechmann et al. (1988) Nature 332:323-327 and Verhoeyen et al. (1988) Science 239:1534-1536), and enzymatic filling-in of gapped oligonucleotides using T₄ DNA polymerase (Queen et al. (1989) Proc. Natl. Acad. Sci. USA 86:10029-10033) can be used to provide molecules having altered or enhanced antigen-binding capabilities and immunogenicity.

Once coding sequences have been prepared or isolated, such sequences can be cloned into any suitable vector or replicon. Numerous cloning vectors are known to those of skill in the art, and the selection of an appropriate cloning vector is a matter of choice. Suitable vectors include, but are not limited to, plasmids, phages, transposons, cosmids, chromosomes or viruses which are capable of replication when associated with the proper control elements.

The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant The coding sequence may or may not contain a signal peptide or leader sequence which can later be removed by the host in post-translational processing. See, e.g., U.S. Pat. Nos. 4,431,739; 4,425,437; 4,338,397.

In addition to control sequences, it may be desirable to add regulatory sequences which allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

As explained above, it may also be desirable to produce mutants or analogs of the polypeptide of interest. Mutants or analogs of HCV polypeptides for use in the subject compositions may be prepared by the deletion of a portion of the sequence encoding the polypeptide of interest, by insertion of a sequence, and/or by substitution of one or more nucleotides within the sequence. Techniques for modifying nucleotide sequences, such as site-directed mutagenesis, and the like, are well known to those skilled in the art See, e.g., Sambrook et al., supra; Kunkel, T. A. (1985) Proc. Natl. Acad. Sci. USA (1985) 82:448; Geisselsoder et al. (1987) BioTechniques 5:786; Zoller and Smith (1983) Methods Enzymol. 100:468; Dalbie-McFarland et al. (1982) Proc. Natl. Acad. Sci USA 79:6409.

The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Nucleic acid molecules comprising nucleotide sequences of interest can be stably integrated into a host cell genome or maintained on a stable episomal element in a suitable host cell using various gene delivery techniques well known in the art. See, e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media If it is not secreted, it can be isolated from cell lysates. The selection of the appropriate growth conditions and recovery methods are within the skill of the art.

The above methods of recombinant production can be used to obtain other polypeptides, such as other HCV polypeptides described below, for administration with the E1E2 compositions.

Microparticles

As explained above, E1E2₈₀₉ DNA is adsorbed to cationic microparticles prior to delivery. Moreover, microparticles can be used to deliver other HCV protein immunogens, as well as DNA encoding the same. For example, microparticles, either cationic, anionic or uncharged, can also be used in compositions for boosting the immune response, for example, for subsequent delivery of either E1E2 DNA, E1 E2 protein, or for delivery of additional immunogens. If used to deliver protein immunogens, the immunogen may be entrapped within or adsorbed to the microparticle.

The term “microparticle” as used herein, refers to a particle of about 100 nm to about 150 μm in diameter, more preferably about 200 nm to about 30 μm in diameter, and most preferably about 500 nm to about 10 μm in diameter. Preferably, the microparticle will be of a diameter that permits parenteral administration without occluding needles and capillaries. Microparticle size is readily determined by techniques well known in the art, such as photon correlation spectroscopy, laser diffractometry and/or scanning electron microscopy.

Microparticles for use herein will be formed from materials that are sterilizable, non-toxic and biodegradable. Such materials include, without limitation, poly(α-hydroxy acid), polyhydroxybutyric acid, polycaprolactone, polyorthoester, polyanhydride, polyvinyl alcohol and ethylenevinyl acetate. Preferably, microparticles for use with the present invention are derived from a poly(α-hydroxy acid), in particular, from a poly(lactide) (“PLA”) (see, e.g., U.S. Pat. No. 3,773,919) or a copolymer of D,L-lactide and glycolide or glycolic acid, such as a poly(D,L-lactide-co-glycolide) (“PLG” or “PLGA”) (see, e.g., U.S. Pat. No. 4,767,628), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the desired dose of polypeptide and the disorder to be treated. These parameters are discussed more fully below. Biodegradable polymers for manufacturing microparticles useful in the present invention are readily commercially available from, e.g., Boehringer Ingelheim, Germany and Birmingham Polymers, Inc., Birmingham, Ala.

Particularly preferred polymers for use herein are PLA and PLG polymers. These polymers are available in a variety of molecular weights, and the appropriate molecular weight to provide the desired release rate for the polynucleotide or polypeptide in question is readily determined by one of skill in the art. Thus, e.g., for PLA, a suitable molecular weight will be on the order of about 2000 to 250,000. For PLG, suitable molecular weights will generally range from about 10,000 to about 200,000, preferably about 15,000 to about 150,000, and most preferably about 50,000 to about 100,000.

If a copolymer such as PLG is used to form the microparticles, a variety of lactide:glycolide ratios will find use herein and the ratio is largely a matter of choice, depending in part on the rate of degradation desired. For example, a 50:50 PLG polymer, containing 50% DL-lactide and 50% glycolide, will provide a fast resorbing copolymer while 75:25 PLG degrades more slowly, and 85:15 and 90:10, even more slowly, due to the increased lactide component. It is readily apparent that a suitable ratio of lactide:glycolide is easily determined by one of skill in the art based on the nature disorder to be treated. Moreover, mixtures of microparticles with varying lactide:glycolide ratios will find use in the formulations in order to achieve the desired release kinetics. PLG copolymers with varying lactide:glycolide ratios and molecular weights are readily available commercially from a number of sources including from Boehringer Ingelheim, Germany and Birmingham Polymers, Inc., Birmingham, Ala. These polymers can also be synthesized by simple polycondensation of the lactic acid component using techniques well known in the art, such as described in Tabata et al., J. Biomed. Mater. Res. (1988) 22:837-858.

Typically, microparticles when used to deliver E1E2 DNA (or other DNA encoding other HCV immunogens and the like) are prepared such that the DNA is adsorbed on the surface. For protein delivery, the antigen can either be entrapped or adsorbed. Several techniques are known in the art for preparing such microparticles. For example, double emulsion/solvent evaporation techniques, such as described in U.S. Pat. No. 3,523,907 and Ogawa et al., Chem. Pharm. Bull. (1988) 36:1095-1103, can be used herein to make the microparticles. These techniques involve the formation of a primary emulsion consisting of droplets of polymer solution, which is subsequently mixed with a continuous aqueous phase containing a particle stabilizer/surfactant More particularly, a water-in-oil-in-water (w/o/w) solvent evaporation system can be used to form the microparticles, as described by O'Hagan et al., Vaccine (1993) 11:965-969 and Jeffery et al., Pharm. Res. (1993) 10:362. In this technique, the particular polymer is combined with an organic solvent, such as ethyl acetate, dimethylchloride (also called methylene chloride and dichloromethane), acetonitrile, acetone, chloroform, and the like. The polymer will be provided in about a 2-15%, more preferably about a 4-10% and most preferably, a 6% solution, in organic solvent The polymer solution is emulsified using e.g., an homogenizer. The emulsion is then combined with a larger volume of an aqueous solution of an emulsion stabilizer such as polyvinyl alcohol (PVA) or polyvinyl pyrrolidone. The emulsion stabilizer is typically provided in about a 2-15% solution, more typically about a 4-10% solution. The mixture is then homogenized to produce a stable w/o/w double emulsion. Organic solvents are then evaporated.

The formulation parameters can be manipulated to allow the preparation of small (<5 μm) and large (>30μm) microparticles. See, e.g., Jeffery et al., Pharm. Res. (1993) 10:362-368; McGee et al., J. Microencap. (1996). For example, reduced agitation results in larger microparticles, as does an increase in internal phase volume. Small particles are produced by low aqueous phase volumes with high concentrations of PVA. Microparticles can also be formed using spray-drying and coacervation as described in, e.g., Thomasin et al., J. Controlled Release (1996) 41:131; U.S. Pat. No. 2,800,457; Masters, K (1976) Spray Drying 2nd Ed. Wiley, New York; air-suspension coating techniques, such as pan coating and Wurster coating, as described by Hall et al., (1980) The “Wurster Process” in Controlled Release Technologies: Methods, Theory, and Applications (A. F. Kydonieus, ed.), Vol. 2, pp. 133-154 CRC Press, Boca Raton, Florida and Deasy, P. B., Crit. Rev. Ther. Drug Carrier Syst. (1988) S(2):99-139; and ionic gelation as described by, e.g., Lim et al., Science (1980) 210:908-910.

Particle size can be determined by, e.g., laser light scattering, using for example, a spectrometer incorporating a helium-neon laser. Generally, particle size is determined at room temperature and involves multiple analyses of the sample in question (e.g., 5-10 times) to yield an average value for the particle diameter. Particle size is also readily determined using scanning electron microscopy (SEM).

Prior to use of the microparticles, DNA or protein content (e.g., the amount of DNA or protein adsorbed to the microparticle or entrapped therein) may be determined so that an appropriate amount of the microparticles may be delivered to the subject in order to elicit an appropriate immunological response. DNA and protein content of the microparticles can be determined according to methods known in the art, such as by disrupting the microparticles and extracting the entrapped or adsorbed molecules. For example, microparticles can be dissolved in dimethylchloride and the agent extracted into distilled water, as described in, e.g., Cohen et al., Pharm. Res. (1991) 8:713; Eldridge et al., Infect. Immun. (1991) 59:2978; and Eldridge et al., J. Controlled Release (1990)11:205. Alternatively, microparticles can be dispersed in 0.1 M NaOH containing 5% (w/v) SDS. The sample is agitated, centrifuged and the supernatant assayed for the particular agent using an appropriate assay. See, e.g., O'Hagan et al., Int. J Pharm. (1994) 103:37-45.

The particles will preferably comprise from about 0.05% to about 40% (w/w) DNA or polypeptide, such as 0.1% to 30%, e.g., 0.5% . . . 1% . . . 1.5% . . . 2% etc. to 25% (w/w), and even more preferably about 0.5%-4% to about 18%-20% (w/w). The load of DNA or polypeptide in the microparticles will depend on the desired dose and the condition being treated, as discussed in more detail below.

Following preparation, microparticles can be stored as is or freeze-dried for further use. In order to adsorb DNA and/or protein to the microparticles, the microparticle preparation is simply mixed with the molecule of interest and the resulting formulation can again be lyophilized prior to use. Generally, for purposes of the present invention, approximately 1 μg to 100 mg of DNA, such as 10 μg to 5mg, or 100 μg to 500 μg, such as 1 . . . 5 . . . 10 . . . 20 . . . 30 . . . 40 . . . 50 . . . 60 . . . 100 μg and so on, to 500 μg DNA, and any integer within these ranges, will be adsorbed with the microparticles described herein.

One preferred method for adsorbing macromolecules onto prepared microparticles is described in International Publication No. WO 00/050006. Briefly, microparticles are rehydrated and dispersed to an essentially monomeric suspension of microparticles using dialyzable anionic or cationic detergents. Useful detergents include, but are not limited to, any of the various N-methylglucamides (known as MEGAs), such as heptanoyl-N-methylglucamide (MEGA-7), octanoyl-N-methylglucamide (MEGA-8), nonanoyl-N-methylglucamide (MEGA-9), and decanoyl-N-methyl-glucamide (MEGA-10); cholic acid; sodium cholate; deoxycholic acid; sodium deoxycholate; taurocholic acid; sodium taurocholate; taurodeoxycholic acid; sodium taurodeoxycholate; 3-[(3-cholamidopropyl)dimethylammonio]-1-propane-sulfonate (CHAPS); 3-[(3-cholamidopropyl)dimethylammonio]-2-hydroxy-1-propane-sulfonate (CHAPSO); Bdodecyl-N,N-dimethyl-3-ammonio-1-propane-sulfonate (ZWITTERGENT 3-12); NN-bis-(3-D-gluconeamidopropyl)-deoxycholamide (DEOXY-BIGCHAP); Boctylglucoside; sucrose monolaurate; glycocholic acid/sodium glycocholate; laurosarcosine (sodium salt); glycodeoxycholic acid/sodium glycodeoxycholate; sodium dodceyl sulfate (SDS); 3-(trimethylsilyl)-1-propanesulfonic acid (DSS); cetrimide (CTAB, the principal component of which is hexadecyltrimethylammonium bromide); hexadecyltrimethylammonium bromide; dodecyltrimethylammonium bromide; hexadecyltrimethyl-ammonium bromide; tetradecyltrimethylammonium bromide; benzyl dimethyldodecylammonium bromide; benzyl dimethylhexadecylammonium chloride; and benzyl dimethyltetradecylammonium bromide. The above detergents are commercially available from e.g., Sigma Chemical Co., St Louis, Mo. Various cationic lipids known in the art can also be used as detergents. See Balasubramaniam et al., 1996, Gene Ther., 3:163-72 and Gao, X, and L. Huang. 1995, Gene Ther., 2:7110-722.

The microparticle/detergent mixture is then physically ground, e.g., using a ceramic mortar and pestle, until a smooth slurry is formed. An appropriate aqueous buffer, such as phosphate buffered saline (PBS) or Tris buffered saline, is then added and the resulting mixture sonicated or homogenized until the microparticles are fully suspended. The macromolecule of interest, such as E1E2 DNA or polypeptide, is then added to the microparticle suspension and the system dialyzed to remove detergent The polymer microparticles and detergent system are preferably chosen such that the macromolecule of interest will adsorb to the microparticle surface while still maintaining activity of the macromolecule. The resulting microparticles containing surface-adsorbed macromolecule may be washed free of unbound macromolecule and stored as a suspension in an appropriate buffer formulation, or lyophilized with the appropriate excipients, as described further below.

Microparticles manufactured in the presence of charged detergents, such as anionic or cationic detergents, yield microparticles with a charged surface having a net negative or a net positive charge. These microparticles can adsorb a greater variety of molecules. For example, microparticles manufactured with anionic detergents, such as sodium dodceyl sulfate (SDS) or 3-(trimethylsily)-1-propanesulfonic acid (DSS), i.e. PLG/SDS or PLGIDSS microparticles, adsorb positively charged immunogens, such as proteins, and are termed “anionic” herein. Similarly, microparticles manufactured with cationic detergents, such as CTAB, i.e. PLG/CTAB microparticles, adsorb negatively charged macromolecules, such as DNA and are termed “cationic” herein.

Other HCV Polypeptides and Polynucleotides

As explained above, the methods of the present invention may employ other compositions comprising HCV antigens or DNA encoding such antigens. Such compositions can be delivered prior to, subsequent to, or concurrent with the E1E2₈₀₉ DNA compositions, as well as prior to, subsequent to, or concurrent with compositions for boosting the immune response, if used.

The genome of the hepatitis C virus typically contains a single open reading frame of approximately 9,600 nucleotides, which is transcribed into a polyprotein. The full-length sequence of the polyprotein is disclosed in European Publication No. 388,232 and U.S. Pat. No. 6,150,087. As shown in Table 1 and FIG. 1, An HCV polyprotein, upon cleavage, produces at least ten distinct products, in the order of NH₂-Core-E1-E2-p7-NS2-NS3-NS4a-NS4b-NS5a-NS5b-COOH. The core polypeptide occurs at positions 1-191, numbered relative to HCV-1 (see, Choo et al. (1991) Proc. Natl. Acad. Sci. USA 88:2451-2455, for the HCV-1 genome). This polypeptide is further processed to produce an HCV polypeptide with approximately amino acids 1-173. The envelope polypeptides, E1 and E2, occur at about positions 192-383 and 384-746, respectively. The P7 domain is found at about positions 747-809. NS2 is an integral membrane protein with proteolytic activity and is found at about positions 810-1026 of the polyprotein. NS2, either alone or in combination with NS3 (found at about positions 1027-1657), cleaves the NS2-NS3 sissle bond which in turn generates the NS3 N-terminus and releases a large polyprotein that includes both serine protease and RNA helicase activities. The NS3 protease, found at about positions 1027-1207, serves to process the remaining polyprotein. The helicase activity is found at about positions 1193-1657. Completion of polyprotein maturation is initiated by autocatalytic cleavage at the NS3-NS4a junction, catalyzed by the NS3 serine protease. Subsequent NS3-mediated cleavages of the HCV polyprotein appear to involve recognition of polyprotein cleavage junctions by an NS3 molecule of another polypeptide. In these reactions, NS3 liberates an NS3 cofactor (NS4a, found about positions 1658-1711), two proteins (NS4b found at about positions 1712-1972, and NS5a found at about positions 1973-2420), and an RNA-dependent RNA polymerase (NS5b found at about positions 2421-3011).

TABLE 1

| Domain | Approximate Boundaries* |
|---|---|
| C (core) | 1-191 |
| E1 | 192-383 |
| E2 | 384-746 |
| P7 | 747-809 |
| NS2 | 810-1026 |
| NS3 | 1027-1657 |
| NS4a | 1658-1711 |
| NS4b | 1712-1972 |
| NS5a | 1973-2420 |
| NS5b | 2421-3011 |

*Numbered relative to HCV-1. See, Choo et al. (1991) Proc. Natl. Acad. Sci. USA 88:2451-2455.

Sequences for the above HCV polyprotein products, DNA encoding the same and immunogenic polypeptides derived therefrom, are known (see, e.g., U.S. Pat. No. 5,350,671). For example, a number of general and specific immunogenic polypeptides, derived from the HCV polyprotein, have been described. See, e.g., Houghton et al., European Publ. Nos. 318,216 and 388,232; Choo et al. Science (1989) 244:359-362; Kuo et al. Science (1989) 244:362-364; Houghton et al. Hepatology (1991) 14:381-388; Chien et al. Proc. Natl. Acad Sci. USA (1992) 89:10011-10015; Chien et al. J. Gastroent. Hepatol. (1993) 8:S33-39; Chien et al., International Publ. No. WO 93/00365; Chien, D.Y., International Publ. No. WO 94/01778. These publications provide an extensive background on HCV generally, as well as on the manufacture and uses of HCV polypeptide immunological reagents.

Any desired immunogenic HCV polypeptide or DNA encoding the same can be utilized with the present invention. For example, HCV polypeptides derived from the Core region, such as polypeptides derived from the region found between amino acids 1-191; amino acids 10-53; amino acids 1045; amino acids 67-88; amino acids 86-100; 81-130; amino acids 121-135; amino acids 120-130; amino acids 121-170; and any of the Core epitopes identified in, e.g., Houghton et al., U.S. Pat. No. 5,350,671; Chien et al. Proc. Natl. Acad. Sci. USA (1992) 89:10011-10015; Chien et al. J. Gastroent. Hepatol. (1993) 8:S33-39; Chien et al., International Publ. No. WO 93/00365; Chien, D.Y., International Publ. No. WO 94/01778; and U.S. Pat. No. 6,150,087, will find use with the subject compositions and methods.

Additionally, polypeptides derived from the nonstructural regions of the virus will also find use herein. The NS3/4a region of the HCV polyprotein has been described and the amino acid sequence and overall structure of the protein are disclosed in Yao et al. Structure (November 1999) 7:1353-1363. See, also, Dasmahapatra et al., U.S. Pat. No. 5,843,752. As explained above, either the native sequence or immunogenic analogs can be used in the subject formulations. Dasmahapatra et al., U.S. Pat. No. 5,843,752 and Zhang et al., U.S. Pat. No. 5,990,276, both describe analogs of NS3/4a and methods of making the same.

Moreover, polypeptides for use in the subject compositions and methods may be derived from the NS3 region of the HCV polyprotein. A number of such polypeptides are known, including, but not limited to polypeptides derived from the c33c and c100 regions, as well as fusion proteins comprising an NS3 epitope, such as c25. These and other NS3 polypeptides are useful in the present compositions and are known in the art and described in, e.g., Houghton et al, U.S. Pat. No. 5,350,671; Chien et al. Proc. Natl. Acad. Sci. USA (1992) 89:10011-10015; Chien et al. J. Gastroent. Hepatol. (1993) 8:S33-39; Chien et al., International Publ. No. WO 93/00365; Chien, D. Y., International Publ. No. WO 94/01778; and U.S. Pat. No. 6,150,087.

Additionally, multiple epitope fusion antigens (termed “MEFAs”), as described in, e.g., U.S. Pat. Nos. 6,514,731 and 6,428,792, may be used in the subject compositions. Such MEFAs include multiple epitopes derived from two or more of the various viral regions. The epitopes are preferably from more than one HCV strain, thus providing the added ability to protect against multiple strains of HCV in a single vaccine.

As explained above, for convenience, the various HCV regions have been defined with respect to the amino acid number relative to the polyprotein encoded by the genome of HCV-1a, as described in Choo et al. (1991) Proc Natl Acad Sci USA 88: 2451, with the initiator methionine being designated position 1. However, HCV polypeptides and polynucleotides for use with the present invention are not limited to those derived from the HCV-1a sequence and any strain or isolate of HCV can serve as the basis for providing antigenic sequences for use with the invention, as explained in detail above.

The above polynucleotides and polypeptides can be obtained using the methods of recombinant production described above for E1E2 polypeptides and polynucleotides.

Immunogenic Compositions and Administration

A. Compositions

Once produced, the E1E2 polynucleotides, polypeptides or other immunogens may be provided in immunogenic compositions, in e.g., prophylactic (i.e., to prevent infection) or therapeutic (to treat HCV following infection) vaccine compositions. The compositions will generally include one or more “pharmaceutically acceptable excipients or vehicles” such as water, saline, glycerol, ethanol, etc. Additionally, auxiliary substances, such as wetting or emulsifying agents, pH buffering substances, and the like, may be present in such vehicles.

A carrier is optionally present, e.g., in protein compositions used to boost the immune response to the E1E2₈₀₉ DNA. Carriers are molecules that do not themselves induce the production of antibodies harmful to the individual receiving the composition. Suitable carriers are typically large, slowly metabolized macromolecules such as proteins, polysaccharides, polylactic acids, polyglycollic acids, polymeric amino acids, amino acid copolymers, lipid aggregates (such as oil droplets or liposomes), and inactive virus particles. Such carriers are well known to those of ordinary skill in the art. Furthermore, the immunogenic polypeptide may be conjugated to a bacterial toxoid, such as toxoid from diphtheria, tetanus, cholera, etc.

Adjuvants may also be present in the compositions to enhance the immune response, such as but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides (see below) or bacterial cell wall components), such as for example (a) MF59 (PCT Publ. No. WO 90/14837; U.S. Pat. Nos. 6,299,884 and 6,451,325), containing 5% Squalene, 0.5% Tween 80, and 0.5% Span 85 (optionally containing various amounts of MTP-PE ), formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as QS21 or Stimulon™ (Cambridge Bioscience, Worcester, Mass.) may be used or particles generated therefrom such as ISCOMs (immunostimulating complexes), which ISCOMs may be devoid of additional detergent (see, e.g., International Publication No. WO 00/07621); (4) Complete Freunds Adjuvant (CFA) and Incomplete Freunds Adjuvant (IFA); (5) cytokines, such as interleukins, such as IL-1, IL-2, IL-4, IL-5, IL-6, IL-7, IL-12 etc. (see, e.g., International Publication No. WO 99/44636), interferons, such as gamma interferon, macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S 109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); (7) monophosporyl lipid A (MPL) or 3-O-deacylated MPL (3dMPL) (see, e.g., GB 2220221; EPA 0689454), optionally in the substantial absence of alum (see, e.g., International Publication No. WO 00/56358); (8) combinations of 3dMPL with, for example, QS21 and/or oil-in-water emulations (see, e.g., EPA 0835318; EPA 0735898; EPA 0761231); (9) a polyoxyethylene ether or a polyoxyethylene ester (see, e.g., International Publication No. WO 99/52549); (10) a saponin and an immunostimulatory oligonucleotide, such as a CpG oligonucleotide (see, e.g., International Publication No. WO 00/62800); (11) an immunostimulant and a particle of a metal salt (see, e.g., International Publication No. WO 00/23105); (12) a saponin and an oil-in-water emulsion (see, e.g., International Publication No. WO 99/11241; (13) a saponin (e.g., QS21)+3dMPL+IL-12 (optionally+a sterol) (see, e.g., International Publication No. WO 98/57659); and (14) other substances that act as immunostimulating agents to enhance the effectiveness of the composition.

Muramyl peptides include, but are not limited to N-acetyl-muramyl-L-threonyl-D-isoglutamine (thr-MDP), N-acteyl-normuramyl-L-alanyl-D-isogluatme (nor-MDP), N-acetylmuramyl-L-alanyl-D-isogluatminyl-L-alanine-2-(1′-2′-dipalmitoyl-sn-glycero-3-hydroxyphosphoryloxy)-ethylamine (MTP-PE), etc.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsions containing 4-5% w/v squalene, 0.25-1.0% w/v Tween 80™ (polyoxyelthylenesorbitan monooleate), and/or 0.25-1.0% Span ₈₅™ (sorbitan trioleate), and optionally, N-acetylmuramyl-L-alanyl-D-isogluatminyl-L-alanine-2-(1′-2′-dipalmitoyl-sn-glycero-3-huydroxyphosphoryloxy)-ethylamine (MTP-PE), for example, the submicron oil-in-water emulsion known as “MF59” (International Publication No. WO 90/14837; U.S. Pat. Nos. 6,299,884 and 6,451,325; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g., 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO 90/14837 and U.S. Pat. Nos. 6,299,884 and 6,451,325.

Other preferred agents to include in the subject compositions are immunostimulatory molecules such as immunostimulatory nucleic acid sequences (ISS), including but not limited to, unmethylated CpG motifs, such as CpG oligonucleotides. Oligonucleotides containing unmethylated CpG motifs have been shown to induce activation of B cells, NK cells and antigen-presenting cells (APCs), such as monocytes and macrophages. See, e.g., U.S. Pat. No. 6,207,646. Thus, adjuvants derived from the CpG family of molecules, CpG dinucleotides and synthetic oligonucleotides which comprise CpG motifs (see, e.g., Krieg et al. Nature (1995) 374:546 and Davis et al. J. Immunol. (1998) 160:870-876) such as any of the various immunostimulatory CpG oligonucleotides disclosed in U.S. Pat. No. 6,207,646, may be used in the subject methods and compositions. Such CpG oligonucleotides generally comprise at least 8 up to about 100 basepairs, preferably 8 to 40 basepairs, more preferably 15-35 basepairs, preferably 15-25 basepairs, and any number of basepairs between these values. For example, oligonucleotides comprising the consensus CpG motif, represented by the formula 5′-X₁CGX₂-3′, where X₁ and X₂ are nucleotides and C is unmethylated, will find use as immunostimulatory CpG molecules. Generally, X₁ is A, G or T, and X₂ is C or T. Other useful CpG molecules include those captured by the formula 5′-X₁X₂CGX₃X₄, where X₁ and X₂ are a sequence such as GpT, GpG, GpA, ApA, ApT, ApG, CpT, CpA, CpG, TpA, TpT or TpG, and X₃ and X4 are TpT, CpT, ApT, ApG, CpG, TpC, ApC, CpC, TpA, ApA, GpT, CpA, or TpG, wherein “p” signifies a phosphate bond. Preferably, the oligonucleotides do not include a GCG sequence at or near the 5′- and/or 3′ terminus. Additionally, the CpG is preferably flanked on its 5′-end with two purines (preferably a GpA dinucleotide) or with a purine and a pyrimidine (preferably, GpT), and flanked on its 3′-end with two pyrimidines, preferably a TpT or TpC dinucleotide. Thus, preferred molecules will comprise the sequence GACGTT, GACGTC, GTCGTT or GTCGCT, and these sequences will be flanked by several additional nucleotides. The nucleotides outside of this central core area appear to be extremely amendable to change.

Moreover, the CpG oligonucleotides for use herein may be double- or single-stranded. Double-stranded molecules are more stable in vivo while single-stranded molecules display enhanced immune activity. Additionally, the phosphate backbone may be modified, such as phosphorodithioate-modified, in order to enhance the immunostimulatory activity of the CpG molecule. As described in U.S. Pat. No. 6,207,646, CpG molecules with phosphorothioate backbones preferentially activate B-cells, while those having phosphodiester backbones preferentially activate monocytic (macrophages, dendritic cells and monocytes) and NK cells.

CpG molecules can readily be tested for their ability to stimulate an immune response using standard techniques, well known in the art. For example, the ability of the molecule to stimulate a humoral and/or cellular immune response is readily determined using the immunoassays described above. Moreover, the immunogenic compositions can be administered with and without the CpG molecule to determine whether an immune response is enhanced.

Compositions for use in the invention will comprise a therapeutically effective amount of DNA encoding the E1E2 complexes (or a therapeutically effective amount of protein) and any other of the above-mentioned components, as needed. By “therapeutically effective amount” is meant an amount of an protein or DNA encoding the same which will induce an immunological response, preferably a protective immunological response, in the individual to which it is administered. Such a response will generally result in the development in the subject of an antibody-mediated and/or a secretory or cellular immune response to the composition. Usually, such a response includes but is not limited to one or more of the following effects; the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell, and/or cytotoxic T cell and/or γδT cell populations.

E1E2 protein compositions, e.g., used to boost the immune response following administration of E1E2₈₀₉ DNA, can comprise mixtures of one or more of the E1E2 complexes, such as E1E2 complexes derived from more than one viral isolate, as well as additional HCV antigens. Moreover, as explained above, the E1E2 complexes may be present as a heterogeneous mixture of molecules, due to clipping and proteolytic cleavage. Thus, a composition including E1E2 complexes may include multiple species of E1E2, such as E1E2 terminating at amino acid 746 (E1E2₇₄₆), E1E2 terminating at amino acid 809 (E1E2₈₀₉), or any of the other various E1 and E2 molecules described above, such as E2 molecules with N-terminal truncations of from 1-20 amino acids, such as E2 species beginning at amino acid 387, amino acid 402, amino acid 403, etc.

The compositions (both DNA and protein) may be administered in conjunction with other antigens and immunoregulatory agents, for example, immunoglobulins, cytokines, lymphokines, and chemokines, including but not limited to cytokines such as IL-2, modified IL-2 (cys125 to ser125), GM-CSF, IL-12, γ-interferon, IP-10, MIP1β, FLP-3, ribavirin and RANTES.

B. Administration

Typically, the immunogenic compositions (both DNA and protein) are prepared as injectables, either as liquid solutions or suspensions; solid forms suitable for solution in, or suspension in, liquid vehicles prior to injection may also be prepared. Thus, once formulated, the compositions are conventionally administered parenterally, e.g., by injection, either subcutaneously or intramuscularly. Additional formulations suitable for other modes of administration include oral and pulmonary formulations, suppositories, and transdermal applications. Dosage treatment may be a single dose schedule or a multiple dose schedule. Preferably, the effective amount is sufficient to bring about treatment or prevention of disease symptoms. The exact amount necessary will vary depending on the subject being treated; the age and general condition of the individual to be treated; the capacity of the individual's immune system to synthesize antibodies; the degree of protection desired; the severity of the condition being treated; the particular macromolecule selected and its mode of administration, among other factors. An appropriate effective amount can be readily determined by one of skill in the art A “therapeutically effective amount” will fall in a relatively broad range that can be determined through routine trials using in vitro and in vivo models known in the art The amount of E1E2 DNA and polypeptides used in the examples below provides general guidance which can be used to optimize the elicitation of anti-E1, anti-E2 and/or anti-E1E2 antibodies.

For example, the immunogen is preferably injected intramuscularly to a large mammal, such as a primate, for example, a baboon, chimpanzee, or human. The amount of E1E2 DNA adsorbed to the cationic microparticles will generally be about 1 μg to 500 mg of DNA, such as 5 μg to 100 mg of DNA, e.g., 10 μg to 50 mg, or 100 μg to 5 mg, such as 20 . . . 30 . . . 40 . . . 50 . . . 60 . . . 100 . . . 200 μg and so on, to 500 μg DNA, and any integer between the stated ranges. The E1E2 expression constructs of the present invention are administered using standard gene delivery protocols. Methods for gene delivery are known in the art. See, e.g., U.S. Pat. Nos. 5,399,346, 5,580,859, 5,589,466. E1E2₈₀₉ DNA can be delivered either directly to the vertebrate subject or, alternatively, delivered ex vivo, to cells derived from the subject and the cells reimplanted in the subject.

Administration of DNA encoding E1E2 polypeptides can elicit a cellular immune response, and/or an anti-E1, anti-E2 and/or anti-E1E2 antibody titer in the mammal that lasts for at least 1 week, 2 weeks, 1 month, 2 months, 3 months, 4 months, 6 months, 1 year, or longer. E1E2 DNA can also be administered to provide a memory response. If such a response is achieved, antibody titers may decline over time, however exposure to the HCV virus or immunogen results in the rapid induction of antibodies, e.g., within only a few days. Optionally, antibody titers can be maintained in a mammal by providing one or more booster injections of the E1E2 polypeptides, as explained above, at 2 weeks, 1 month, 2 months, 3 months, 4 months, 5 months, 6 months, 1 year, or more after the primary injection.

Preferably, an antibody titer of at least 10, 100, 150, 175, 200, 300, 400, 500, 750, 1,000, 1,500, 2,000, 3,000, 5,000, 10,000, 20,000, 30,000, 40,000, 50,000 (geometric mean titer), or higher, is elicited, or any number between the stated titer, as determined using a standard immunoassay, such as the immunoassay described in the examples below. See, e.g., Chien et al., Lancet (1993) 342:933; and Chien et al., Proc. Natl. Acad. Sci. USA (1992) 89:10011.

For an E1E2 protein boost, generally about 0.1 μg to about 5.0 mg of immunogen will be delivered per dose, or any amount between the stated ranges, such as 0.5 μg to about 10 mg, 1 μg to about 2 mg, 2.5 μg to about 250 μg, 4 μg to about 200 μg, such as 4, 5, 6, 7, 8, 9, 10 . . . 20 . . . 30 . . . 40 . . . 50 . . . 60 . . . 70 . . . 80 . . . 90 . . . 100, etc., μg per dose. The immunogens can be administered either to a mammal that is not infected with an HCV or can be administered to an HCV-infected mammal.

Deposits of Strains Useful in Practicing the Invention

A deposit of biologically pure cultures of the following strains was made with the American Type Culture Collection, 10801 University Boulevard, Manassas, Va. The accession number indicated was assigned after successful viability testing, and the requisite fees were paid, made under the provisions of the Budapest Treaty on the International Recognition of the Deposit of Microorganisms for the Purpose of Patent Procedure and the Regulations thereunder (Budapest Treaty). This assures maintenance of viable cultures for a period of thirty (30) years from the date of deposit The organisms will be made available by the ATCC under the terms of the Budapest Treaty, which assures permanent and unrestricted availability of the progeny to one determined by the U.S. Commissioner of Patents and Trademarks to be entitled thereto according to 35 U.S.C. §122 and the Commissioner's rules pursuant thereto (including 37 C.F.R. § 1.12 with particular reference to 886 OG 638). Upon the granting of a patent, all restrictions on the availability to the public of the deposited cultures will be irrevocably removed.

These deposits are provided merely as convenience to those of skill in the art, and are not an admission that a deposit is required under 35 U.S.C. § 112. The nucleic acid sequences of these genes, as well as the amino acid sequences of the molecules encoded thereby are controlling in the event of any conflict with the description herein. A license may be required to make, use, or sell the deposited materials, and no such license is hereby granted.

| Plasmid | Deposit Date | ATCC No. |
|---|---|---|
| E1E2-809 | Aug. 16, 2001 | PTA-3643 |

### 2. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

Enzymes were purchased from commercial sources, and used according to the manufacturers' directions.

In the isolation of DNA fragments, except where noted, all DNA manipulations were done according to standard procedures. See, Sambrook et al., supra. Restriction enzymes, T₄ DNA ligase, E. coli, DNA polymerase 1I, Klenow fragment, and other biological reagents can be purchased from commercial suppliers and used according to the manufacturers' directions. Double stranded DNA fragments were separated on agarose gels.

Sources for chemical reagents generally include Sigma Chemical Company, St. Louis, Mo.; Alrich, Milwaukee, Wis.; Roche Molecular Biochemicals, Indianapolis, Ind.

Plasmid Design.

The plasmid pCMVtpaE1E2p7 (6275 bp) was constructed by cloning HCV-1 encoding amino acids 192 to 809 with the upstream tissue plasminogen activator (tpa) signal sequence into the pnewCMV-II expression vector. The pnewCMV vector is a pUC19-based cloning vector comprising the following elements: an SV40 origin of replication, a human CMV enhancer/promoter, a human CMV intron, a human tissue plasminogen activator (tPA) leader, a bovine growth hormone poly A terminator and an ampicillin resistance gene.

E1E2₈₀₉ was expressed from recombinant CHO cells as described previously (Spaete et al., Virology (1992) 188:819-830). E1E2 antigen was extracted from inside the CHO cells with Triton X-100 detergent. The E1E2 antigen was purified using Galanthus nivalis lectin agarose (Vector Laboratories, Burlingame, Calif.) chromatography and fast flow S-Sepharose cation-exhange chromatography (Pharmacia). The oil-in-water adjuvant MF59 was manufactured at Chiron Vaccines, Marburg and has previously been described in detail (Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296)

For the CTL assays, fifty-four peptides (each 20 amino acids in length overlapping by 10 amino acids) spanning the E1 and E2 proteins (amino acids 192-809) of HCV-1a were synthesized with free amine N-termini and free acid C-termini by Chiron Mimotopes Pty. Ltd. (Clayton, Australia). The lyophilized peptides were resuspended in 10% DMSO in water, and then each was diluted to 2 mg/ml. Using equal volumes of each peptide, 2 pools of 27 peptides each were made: Pool 1 (amino acids 192-470) and Pool 2 (amino acids 461-740). The recombinant vaccinia virus (VV) expressing HCV-1a amino acids 134-966 (Sc59 E12C/B) was generated by methods previously described (Choo et al., Proc. Natl. Acad. Sci. USA (1994) 91:1294-1298). U96-Nunc Maxisorp plates (Nalgene Nunc International Rochester, N.Y.), Goat anti-Mouse IgG-HRP conjugate (Caltag Laboratories, Burlingame, Calif.), and TMB Microwell Peroxidase Substrate System (Kirkegaard & Perry Laboratories, Gaithersburg, Md.) were used for the ELISA.

Polylactide-co-glycolide (RG 504, 50:50 lactide:glycolide monomer ratio) was obtained from Boehringer Ingelheim, USA. CTAB was obtained from Sigma Chemical Co., St Louis, U.S.A. and was used as shipped. PLG/CTAB microparticles were prepared using a solvent evaporation technique essentially as described previously (Singh et al., Proc. Natl. Acad. Sci. USA (2000) 97:811-816; Briones et al., Pharm. Res. (2001) 18:709-712). The HCV E1E2 plasmid was adsorbed onto the microparticles by incubating 100 mg of microparticles with a 200 μg/ml solution of DNA in 1× TE buffer under gentle stirring at 4° C. for 12 hours. The microparticles were then separated by centrifugation, followed by lyophilization. The amount of adsorbed DNA was determined by hydrolysis of the PLG microparticles. The size distribution of the microparticles was determined using a particle size analyzer (Malvern Instruments, Malvern, U.K.). The zeta potential was measured on a DELSA 440 SX Zetasizer (Coulter Corp. Miami, Fla.).

### Example 1

### Immunization of Mice Using E1E2 DNA Adsorbed to Cationic Microparticles

Three studies on mice were conducted to determine the immunogenicity of E1E2₈₀₉ plasmid DNA adsorbed to cationic microparticles. In the first study, groups of 10 female CB6F1 mice age 6-8 weeks and weighing about 20-25 g were immunized with E1E2₈₀₉ plasmid DNA or PLG/CTAB/ E1E2₈₀₉DNA (10 and 100 μg) at days 0 and 28. The formulations were injected in saline by the TA route in the two hind legs (50 μl per site) of each animal. Mice were bled on day 42 through the retro-orbital plexus and the sera were separated. HCV E1E2-specific serum IgG titers were quantified by ELISA.

In the second study, immunization with 1 and 10 μg of PLG/CTAB/E1E2₈₀₉DNA was compared to immunization with 2 μg of recombinant E1E2₈₀₉ protein in MF59 at 0 and 28 days, in groups of 10 mice each. An additional group of mice was immunized with 10 μg of E1E2₈₀₉ plasmid DNA for comparison and sera was separated for assay on day 42.

In the third mice study, immune responses elicited by E1E2₈₀₉ plasmid DNA, PLG/CTAB/ E1E2₈₀₉DNA and DNA prime/protein boost were compared. The initial immunizations were done with E1E2₈₀₉ plasmid DNA (10 μg), PLG/CTAB/E1E2₈₀₉DNA (10 μg) or 5 μg of E1E2₈₀₉ protein in MF59. Three groups of 10 mice each were immunized three times exclusively with PLG/CTAB/E1E2₈₀₉DNA, E1E2₈₀₉ plasmid DNA, or E1E2₈₀₉ protein in MF59. In addition, two further groups of mice received two doses of either PLG/CTAB/E1E2₈₀₉DNA or E1E2₈₀₉plasmid DNA (10 μg), and both groups were boosted with a third immunization, consisting of a single dose of E1E2₈₀₉ protein (5 μg) in MF59. All groups of animals were immunized on three occasions, separated by four weeks and sera was collected on day 70.

The antibody responses against HCV E1E2 in mice were measured on the sera collected two weeks after each immunization by ELISA. Microtiter plates were coated with 200 μl of the purified HCV E1E2₈₀₉ at 0.625 μg/ml overnight at 4° C. The coated wells were blocked for 1 hr at 37° C. with 300 μl of 1% BSA in phosphate-buffered saline (PBS). The plates were washed five times with a washing buffer (PBS, 0.3% Tween-20), tapped, and dried. Serum samples and a serum standard were initially diluted in the blocking buffer and then transferred into coated, blocked plates in which the samples were serially diluted three-fold with the same buffer. Plates were washed after 1-hour incubation at 37° C. Horseradish peroxidase conjugated goat anti-mouse IgG gamma chain specific (Caltag Laboratories, Inc.) was used to determine the total IgG titer. After the 1-hour incubation at 37° C., plates were washed to remove unbound antibodies. OPD substrate was used to develop the plates, and the color reaction was blocked after 30 minutes by the addition of 4N HCL. The titers of IgG antibodies were expressed as the reciprocal of the sample dilution, in which the optical density of the diluted sample equaled 0.5 at 492 and 620 nm.

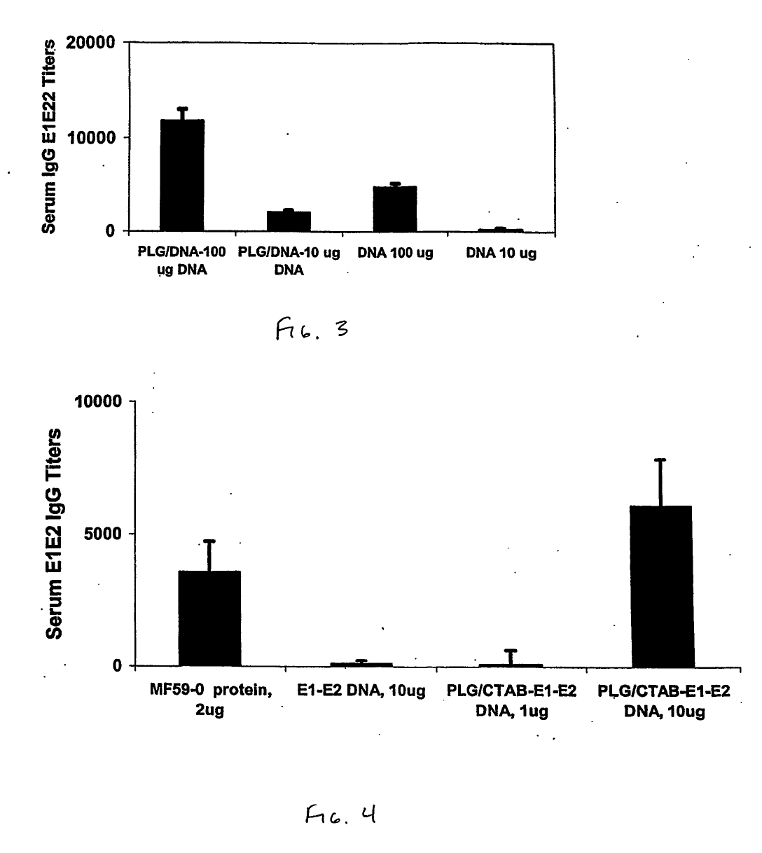

In the first study, significantly enhanced serum IgG antibody responses to E1E2 were induced by adsorbing the E1E2₈₀₉ plasmid DNA to PLG/CTAB microparticles, in comparison to immunization with E1E2₈₀₉ plasmid DNA alone at both doses (10 and 100 μg of DNA). In addition, it was clear that 10 μg of E1E2₈₀₉ plasmid DNA was below the threshold dose needed to induce a detectable response. In contrast, PLG/CTAB/E1E2₈₀₉DNA induced a potent response at 10 μg (FIG. 3).

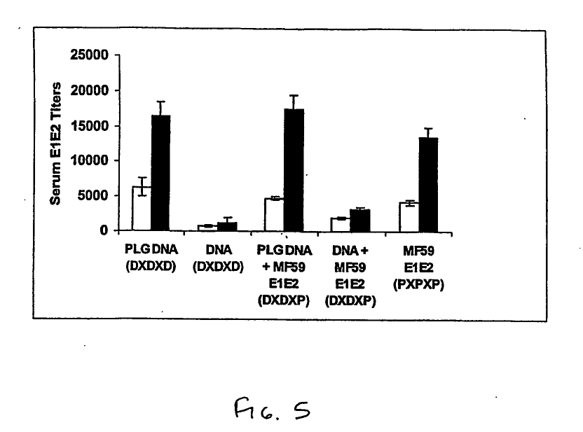

The second study confirmed the ability of PLG/CTAB/E1E2₈₀₉DNA to induce a significantly enhanced response over E1E2₈₀₉ plasmid DNA alone at 10 μg, but also showed that PLG/CTAB/E1E2₈₀₉DNA did not induce a potent response at 1 μg. In addition, this study also showed that PLG/CTAB/E1E2₈₀₉DNA (10 μg) induced a comparable response to 2 μg of E1E2₈₀₉ protein adjuvanted with MF59 (FIG. 4).

The third study confirmed and extended the observations from the earlier studies. PLG/CTAB/E1E2₈₀₉DNA was significantly more potent than E1E2₈₀₉ plasmid DNA alone at 10 μg after two or three doses, and was comparable to immunization with 5 μg E1E2₈₀₉ protein in MF59, after two or three doses. In addition, although three doses of 10 μg of E1E2₈₀₉ plasmid DNA did not induce a detectable response, two doses of PLG/CTAB/E1E2₈₀₉DNA (10 μg) induced a potent response (FIG. 5). Moreover, two doses of PLG/CTAB/E1E2₈₀₉DNA (10 μg ) primed for a potent response following boosting with E1E2₈₀₉ protein in MF59, while E1E2₈₀₉ plasmid DNA alone (10 μg) was less effective as a priming regimen. Furthermore, three doses of PLG/CTAB/E1E2₈₀₉DNA (10 μg ) was equally potent to two doses of PLG/CTAB/E1E2₈₀₉DNA (10 μg), followed by a boost with a single dose of 5 μg E1E2₈₀₉ protein in MF59 (FIG. 5).

As shown herein, the E1E2₈₀₉ plasmid was able to induce detectable titers at a dose of 100 μg in mice. However, the cationic PLG microparticles with adsorbed E1E2₈₀₉ DNA were remarkably more potent and were comparable to the responses induced by immunization with recombinant E1E2₈₀₉ protein adjuvanted with MF59. This is in contrast to a previous study using HCV E2 plasmid in mice (Song et al., J. Virol. (2000) 74:2020-2025). In that study, plasmid DNA, even at a high dose (100 μg) was unable to induce detectable antibody responses and a protein booster dose was required to induce seroconversion. Although the present results are consistent with previous data on HIV plasmids adsorbed to PLG microparticles (O'Hagan et al., J. Virol. (2001) 75:9037-9043), the E1E2₈₀₉ antigen expressed from the plasmid used here is very different from antigens previously evaluated in conjunction with PLG. The env plasmid previously evaluated (Briones et al., Pharm. Res. (2001) 18:709-712; O'Hagan et al., J. Virol. (2001) 75:9037-9043) was codon-optimized for high level expression in mammalian cells, with optimal secretion of antigen (Widera et al., J. Immunol. (2000) 164:4635-4640), while the gag plasmid previously evaluated (Singh et al., Proc. Natl. Acad. Sci. USA (2000) 97:811-816; O'Hagan et al., J. Virol. (2001) 75:9037-9043) was also codon-optimized and is efficiently secreted from cells (Zur Megede et al., J. Virol. (2000) 74:2628-2635). In contrast, the E1E2₈₀₉ plasmid used in the current studies was designed to produce the antigen intracellularly (See, e.g., International Publication No. WO 98/50556). Hence, a surprising observation in the current studies is the ability of the PLG microparticles to induce enhanced antibody responses to an antigen which is not designed to be secreted from the cells.

In the third mouse study, the ability of E1E2₈₀₉ plasmid DNA versus PLG/CTAB/E1E2₈₀₉DNA to prime for a potent antibody response following a boost with recombinant E1E2₈₀₉ protein in MF59 adjuvant was studied. Although E1E2₈₀₉ plasmid DNA was able to prime for a boost response by protein, even three doses of E1E2₈₀₉ plasmid DNA (10 μg) alone could not initiate a primary response. In contrast, two doses of PLG/CTAB/ E1E2₈₀₉DNA (10 μg) induced a potent serum antibody response. In addition, PLG/CTAB/E1E2₈₀₉DNA was also more effective at priming for a boost response to protein than E1E2₈₀₉ plasmid DNA alone. Furthermore, a very surprising observation was that three doses of PLG/CTAB/E1E2₈₀₉DNA were comparable to two doses, followed by a protein boost. On several previous occasions, DNA has been shown to be ineffective at inducing potent antibody responses, but the responses have been significantly enhanced by a protein boost.

### Example 2

### Immunization of Rhesus Macaques Using E1E2 DNA Adsorbed to Cationic Microparticles

Based on the above positive results, the following primate study was conducted. Groups of three rhesus macaques were immunized with PLG/CTAB/E1E2₈₀₉DNA (1 mg), or 50 μg of E1E2₈₀₉ protein in MF59 at weeks 0, 4, 8 and 24. In addition, all animals were boosted with 40 μg of E1E2₈₀₉ protein in MF59 at week 64 (see, Table 2).

TABLE 2
Immunization regimen for two groups of three rhesus
macaques immunized with PLG/CTAB/E1E2₈₀₉DNA,
or E1E2₈₀₉ recombinant protein in MF59.

| | | | Dose | Immunization |
|---|---|---|---|---|
| Group | Animal # | Formulation | (Route) | schedule (weeks) |
| 1 | AY922 | PLG/CTAB/ | 1 mg (IM) | 0, 4, 8, 24 and |
| | BB227 | E1E2₈₀₉DNA | | 64 (40 μg E1E2₈₀₉ |
| | BB230 | | | protein boost) |
| 2 | 15862 | E1E2₈₀₉ | 50 μg (IM) | 0, 4, 8, 24 and |
| | 15863 | protein/MF59 | | 64 (40 μg E1E2₈₀₉ |
| | 15864 | | | protein boost) |

The antibody responses against HCV E1E2 in rhesus macaques were measured following the protocol described above. The only difference was that goat anti-rhesus (Southern Biotech Association, Inc.) was used as secondary antibody.

Peripheral blood was drawn from the femoral vein while the animals were under anesthesia. PBMCs were obtained after centrifugation over a Ficoll-Hypaque gradient and were cultured in 24-well dishes at 5×10⁶ cell/well. Of those cells, 1×10⁶ were sensitized with 10 μM of a peptide pool (consisting of individual peptides) for 1 h at 37° C., washed and added to the remaining 4×10⁶ untreated PBMCs in 2 ml of culture medium (RPMI 1640, 10% heat-inactivated FBS, and 1% antibiotics) supplemented with 10 ng/ml of IL-7 (R&D Systems, Minneapolis, Minn.). After 48 h, 5% (final) IL2-containing supernatant (T-STIM without PHA, Becton Dickinson Biosciences—Discovery Labware, San Jose, Calif.) and 50 U/ml (final) of rIL-2 were added to the cultures. Cultures were fed every 34 days. After 10 days in culture, CD8⁺ T cells were isolated using anti-CD8 Abs bound to magnetic beads (Dynal, Oslo, Norway) according to the manufacturer's instructions. Purified CD8⁺ cells (>93% pure as determined by flow cytometry) were cultured for another 2-3 days before being assayed for cytotoxic activity. B-LCLs were derived from each animal using supernatants from the Herpesvirus papio producer cell line S394.

Cytotoxic activity was assessed in a standard ⁵¹Cr release assay. Autologous B-LCLs were incubated with 9.25 mg/ml peptides and 50 mCi ⁵¹Cr for 1.5 hours, washed three times, and plated into a 96-well plate at 5×10³ cells/well. The CD8⁺ T cells were plated at three effector to target (E:T) cell ratios in duplicate. Effectors and targets were incubated together for 4 hours in the presence of 3.75×10⁵ unlabeled targets per well that were included to minimize lysis of B-LCLs by H. papio and/or endogenous foamy virus-specific CTLs. Supernatants (50 ml) were transferred to Lumaplates (Packard Bioscience, Meriden, Conn.), and radioactivity was measured with a Wallac Microbeta 1450 scintillation instrument (Perkin Elmer, Boston, Mass.). Percent specific lysis was calculated as 100×[(mean experimental release−mean spontaneous release)/(mean maximal release−mean spontaneous release)]. CTL responses were scored as positive when percent specific lysis at the two highest E:T cell ratios was greater than or equal to the percent lysis of control targets plus 10 percent.

All three rhesus immunized with E1E2₈₀₉ protein in MF59 showed serum IgG responses two weeks after the second immunization, which were boosted with a third immunization. Two of the three rhesus immunized with PLG/CTAB/E1E2₈₀₉DNA responded two weeks after the second immunization, and all three animals responded following a third immunization. Therefore, seroconversion was achieved in all three rhesus immunized with PLG/CTAB/ E1E2₈₀₉DNA following a third dose. There was no evidence of boosting for the two responding animals for the third dose, although boosting was seen following the fourth dose of PLG/CTAB/E1E2₈₀₉DNA in all animals (Table 3). This suggested that the third dose of DNA was spaced too close to the second to achieve effective boosting. There was a much greater delay between the third and fourth doses, and boosting was achieved following the fourth dose. Nevertheless, the levels of IgG induced by PLG/CTAB/ E1E2₈₀₉DNA were generally lower than the responses induced by E1E2₈₀₉ protein in MF59 after each immunization. However, a single dose of E1E2₈₀₉ protein induced excellent boosting in rhesus previously immunized with PLG/CTAB/E1E2₈₀₉DNA, while a dose of protein given to the animals previously immunized four times with protein did not induce a similar level of boosting. Hence, following five immunizations, comparable serum antibody responses were achieved in both groups of animals which were immunized with protein alone in MF59, or immunized with PLG/CTAB/E1E2₈₀₉DNA followed by a single booster dose of E1E2₈₀₉ protein in MF59.

Two weeks after the fourth immunization with PLG/CTAB/E1E2₈₀₉DNA, CTL responses from PBMC's were evaluated in all animals. One animal (BB227) out of the three immunized with PLG/CTAB/E1E2₈₀₉DNA showed a peptide-specific CTL response (Table 4). This animal (BB227) was the weakest responder for antibodies and only seroconverted weakly following the third dose of PLG/CTAB/E1E2₈₀₉DNA.

To summarize, PLG/CTAB/E1E2₈₀₉DNA microparticles induced seroconversion in 3/3 animals, following three immunizations, and responses were boosted after a fourth dose. Although there was little boosting of the response to DNA following the third immunization, the third dose did induce seroconversion in the one remaining animal which had not yet responded. Although, the serum IgG responses induced with PLG/CTAB/E1E2₈₀₉DNA were significantly less than the responses induced by the recombinant E1E2₈₀₉ protein in MF59, given the previous poor efficacy of DNA vaccines for the induction of antibody responses in primates even following large doses on multiple occasions (Gurunathan et al., Ann. Rev. Immunol. (2000) 18:927-974), the ability of PLG/CTAB/E1E2₈₀₉DNA to induce seroconversion in rhesus macaques is both striking and encouraging.

Although PLG/CTAB/E1E2₈₀₉DNA alone was not capable of inducing comparable serum IgG responses to immunization with E1E2₈₀₉ protein in MF59, a single booster dose of E1E2₈₀₉ protein significantly enhanced the antibody responses in the PLG/CTAB/E1E2₈₀₉DNA-immunized rhesus. Following a single booster dose with recombinant E1E2₈₀₉ protein in MF59, the PLG/CTAB/E1E2₈₀₉DNA group had comparable serum IgG titers to the rhesus which had been immunized exclusively with E1E2₈₀₉ protein in MF59 on five occasions. Since E1E2₈₀₉ is produced as an intracellular antigenic complex (Heile et al., J. Virol. (2000) 74:6885), it is difficult to manufacture as a recombinant protein at the levels required for a universal HCV vaccine. Therefore, the ability of PLG/CTAB/BlE2₈₀₉DNA to prime an anti-E1E2 response that can be boosted with a single dose of E1E2 protein in MF59 provides a protein dose-sparing option for vaccine development. In addition, DNA vaccines can prime CTL responses which may be important in the protective immune response against HCV. Generally, protein based vaccines have been ineffective for the induction of CTL responses in non-human primates and humans (Singh and O'Hagan, Nat. Biotechnol. (1999) 17:1075-1081). In one of the three rhesus macaques immunized with PLG/CTAB/E1E2₈₀₉DNA, a CTL response was detected following the fourth immunization. Although CTL was not evaluated in the E1E2₈₀₉/MF59 immunized animals, the inventors herein have sufficient experience with this adjuvant to be confident that a CTL response would not have been induced.

TABLE 3
Serum IgG antibody responses in rhesus macaques immunized with
PLG/CTAB/E1E2₈₀₉DNA or E1E2₈₀₉ protein in MF59.

| | E2 antibody titers | | | | | |
|---|---|---|---|---|---|---|
| | Animal immunized with | | | | | |
| | E1E2₈₀₉ + MF59 | | | PLG/CTAB/E1E2₈₀₉DNA | | |
| | 15862 | 15863 | 15864 | AY922 | BB227 | BB230 |
| Pre | <5 | <5 | <5 | <5 | <5 | <5 |
| 2w post 1st | NT | NT | NT | <5 | <5 | <5 |
| 2w post 2nd | 550 | 638 | 538 | 150 | <5 | 75 |
| 2w post 3rd | 988 | 763 | 2488 | 125 | 25 | 75 |
| 14w post 3rd | 113 | 50 | 250 | <5 | <5 | <5 |
| 2w post 4th | 813 | 625 | 6525 | 375 | 63 | 375 |
| 40w post 4th | 25 | 13 | 188 | <5 | <5 | <5 |
| 2w post 5th | 475 | 575 | 1388 | 925 | 363 | 3075 |

TABLE 4
Cytotoxic T lymphocyte response in rhesus macaque immunized with
PLG/CTAB/E1E2₈₀₉DNA two weeks after the fourth immunization.
Percent specific lysis at different effector/target cell ratios.

| Effector/ | Unsensitized | Percent lysis with pool 1- |
|---|---|---|
| Target cell ratio | controls | sensitized targets |
| 40/1 | 5 | 24 |
| 13/1 | <1 | 14 |
| 4/1 | <1 | 12 |

### Example 3

### Immunization of Chimpanzees Using E1E2 DNA Adsorbed to Cationic Microparticles

Groups of chimpanzees were immunized in each thigh as shown in Tables 5 and 6, with 3mg (per thigh) of a mixture of plasmids as follows: PLG/CTAB/E1E2₈₀₉DNA, PLG/CTAB/HCV NS34a, PLG/CTAB/HCV NS4aNS4b and PLG/CTAB/HCV NS5. Control animals were not given a vaccine. At month 6, chimps were challenged intravenously with 100 CID of HCV-H strain.

As shown in the tables, PLG DNA primed anti-E1E2 antibodies. Additionally, following challenge, the vaccinated animals became viremic but 4/5 of the animals that were administered PLG/CTAB/E1E2₈₀₉DNA eventually recovered and did not progress to the carrier state which in humans is accompanied with the major pathogenic effects of HCV. In contrast, out of a total of 14 controls challenged with HCV-H, only 6/14 were able to clear the viral infection. These data demonstrate that E1E2 DNA, adsorbed to cationic microparticles, exhibits a prophylactic effect

Moreover, following challenge, there was evidence of a more rapid influx of HCV-specific T cells into the livers of the animals administered PLG/CTAB/E1E2₈₀₉DNA versus the controls, thus further demonstrating the effectiveness of E1E2 DNA adsorbed to cationic microparticles.

Thus, E1E2₈₀₉ DNA compositions and methods of using the same are described. Although preferred embodiments of the subject invention have been described in some detail, it is understood that obvious variations can be made without departing from the spirit and the scope of the invention as defined by the claims herein.

TABLE 5
Elisa for antibody titer against CHO E1/E2

| CHIMP | Date | Treatment | OD | Dlln | Titer |
|---|---|---|---|---|---|
| 4x0179 | (Wk 0) | Control | 0.049 | 40 | — |
| 4x0195 | | | 0.048 | 40 | — |
| 4x0197 | | | 0.024 | 40 | — |
| 4x0320 | | | 0.024 | 40 | — |
| 4x0397 | | | 0.026 | 40 | — |
| 4x0179 | (Wk 4) | Control | 0.037 | 40 | — |
| 4x0195 | | | 0.069 | 40 | — |
| 4x0197 | | | 0.029 | 40 | — |
| 4x0320 | | | 0.039 | 40 | — |
| 4x0397 | | | 0.045 | 40 | — |
| 4x0179 | (Wk 8) | Control | 0.030 | 40 | — |
| 4x0195 | | | 0.050 | 40 | — |
| 4x0197 | | | 0.032 | 40 | — |
| 4x0320 | | | 0.040 | 40 | — |
| 4x0397 | | | 0.047 | 40 | — |
| 4x0179 | (Wk 12) | Control | 0.026 | 40 | — |
| 4x0195 | | | 0.053 | 40 | — |
| 4x0197 | | | 0.037 | 40 | — |
| 4x0320 | | | 0.025 | 40 | — |
| 4x0397 | | | 0.026 | 40 | — |
| 4x0179 | (Wk 16) | Control | 0.017 | 40 | — |
| 4x0195 | | | 0.050 | 40 | — |
| 4x0197 | | | 0.030 | 40 | — |
| 4x0320 | | | 0.028 | 40 | — |
| 4x0397 | | | 0.018 | 40 | — |
| 4x0179 | (Wk 22) | Control | 0.058 | 40 | — |
| 4x0195 | | | 0.034 | 40 | — |
| 4x0197 | | | 0.036 | 40 | — |
| 4x0320 | | | 0.042 | 40 | — |
| 4x0397 | | | 0.043 | 40 | — |
| 4x0179 | (Wk 28) | Control | 0.035 | 40 | — |
| 4x0195 | | | 0.041 | 40 | — |
| 4x0197 | | | 0.033 | 40 | — |
| 4x0320 | | | 0.051 | 40 | — |
| 4x0397 | | | 0.031 | 40 | — |

TABLE 6
Elisa for antibody titer against CHO E1/E2

| CHIMP | Date | Treatment | OD | Dlln | Titer | GM+/−SE |
|---|---|---|---|---|---|---|
| 4x0238 | (Wk −3) | PLG DNA | 0.187 | 40 | — | 1.7+/−0.9 |
| 4x0239 | | | 0.341 | 40 | 14 | |
| 4x0250 | | | 0.146 | 40 | — | |
| 4x0278 | | | 0.145 | 40 | — | |
| 4x0288 | | | 0.117 | 40 | — | |
| 4x0238 | (Wk 0) | PLG DNA | 0.139 | 40 | — | 3.4+/−2.5 |
| 4x0239 | | | 0.497 | 40 | 20 | |
| 4x0250 | | | 0.513 | 40 | 21 | |
| 4x0278 | | | 0.194 | 40 | — | |
| 4x0288 | | | 0.167 | 40 | — | |
| 4x0238 | (Wk 4) | PLG DNA | 0.317 | 40 | 13 | 14.9+/−18.6 |
| 4x0239 | | | 0.594 | 800 | 475 | |
| 4x0250 | | | 0.602 | 200 | 120 | |
| 4x0278 | | | 0.184 | 40 | — | |
| 4x0288 | | | 0.150 | 40 | — | |
| 4x0238 | (Wk 8) | PLG DNA | 0.691 | 40 | 28 | 28.5+/−29.2 |
| 4x0239 | | | 0.529 | 800 | 423 | |
| 4x0250 | | | 0.569 | 200 | 114 | |
| 4x0278 | | | 0.355 | 40 | 14 | |
| 4x0288 | | | 0.136 | 40 | — | |
| 4x0238 | (Wk 12) | PLG DNA | 0.685 | 40 | 27 | 17.8+/−14.6 |
| 4x0239 | | | 0.751 | 200 | 150 | |
| 4x0250 | | | 0.843 | 40 | 34 | |
| 4x0278 | | | 0.334 | 40 | 13 | |
| 4x0288 | | | 0.131 | 40 | — | |
| 4x0238 | (Wk 16) | PLG DNA | 0.531 | 40 | 21 | 9.3+/−8.9 |
| 4x0239 | | | 0.571 | 200 | 114 | |
| 4x0250 | | | 0.722 | 40 | 29 | |
| 4x0278 | | | 0.236 | 40 | — | |
| 4x0288 | | | 0.131 | 40 | — | |
| 4x0238 | (Wk 22) | PLG DNA | 0.370 | 40 | 15 | 5.9+/−4.3 |
| 4x0239 | | | 0.684 | 40 | 27 | |
| 4x0250 | | | 0.455 | 40 | 18 | |
| 4x0278 | | | 0.196 | 40 | — | |
| 4x0288 | | | 0.082 | 40 | — | |
| 4x0238 | (Wk 27) | PLG DNA | 0.248 | 40 | — | 3.4+/−2.6 |
| 4x0239 | | | 0.567 | 40 | 23 | |
| 4x0250 | | | 0.509 | 40 | 20 | |
| 4x0278 | | | 0.165 | 40 | — | |
| 4x0288 | | | 0.094 | 40 | — | |

We claim:

1. A composition consisting essentially of a pharmaceutically acceptable excipient and a polynucleotide adsorbed to a cationic microparticle, wherein said polynucleotide comprises a coding sequence that encodes a hepatitis C virus (HCV) immunogen operably linked to control elements that direct the transcription and translation of said coding sequence in vivo, and further wherein the HCV immunogen is an immunogenic HCV E1E2 complex with a contiguous sequence of amino acids having at least 80% sequence identity to the contiguous sequence of amino acids depicted at positions 192-809 of FIGS. 2A-2C, with the proviso that said polynucleotide does not encode an HCV immunogen other than the HCV E1E2 complex.

2. The composition of claim 1, wherein said HCV E12 complex consists of the sequence of amino acids depicted at positions 192-809 of FIGS. 2A-2C.

3. The composition of claim 1, wherein the cationic microparticle is formed from a polymer selected from the group consisting of a poly(α-hydroxy acid), a polyhydroxy butyric acid, a polycaprolactone, a polyorthoester, and a polyanhydride.

4. The composition of claim 3, wherein the cationic microparticle is formed from a poly(α-hydroxy acid) selected from the group consisting of poly(L-lactide), poly(D,L-lactide) and poly(D,L-lactide-co-glycolide).

5. The composition of claim 4, wherein the cationic microparticle is formed from poly(D,L-lactide-co-glycolide).

6. A composition consisting essentially of:
(a) a pharmaceutically acceptable excipient; and
(b) a polynucleotide adsorbed to a cationic microparticle formed from poly(D,L-lactide-co-glycolide), wherein said polynucleotide comprises a coding sequence that encodes a hepatitis C virus (HCV) immunogen operably linked to control elements that direct the transcription and translation of said coding sequence in vivo, and further wherein the HCV immunogen is an HCV E1E2 complex consisting of the sequence of amino acids depicted at positions 192-809 of FIGS. 2A-2C, with the proviso that said polynucleotide does not encode an HCV immunogen other than the HCV E1E2 complex.

7. A method of stimulating an immune response in a vertebrate subject which comprises administering to the subject a therapeutically effective amount of a first composition consisting essentially of a pharmaceutically acceptable excipient and a polynucleotide adsorbed to a cationic microparticle, wherein said polynucleotide comprises a coding sequence that encodes a hepatitis C virus (HCV) immunogen operably linked to control elements that direct the transcription and translation of said coding sequence in vivo, and further wherein the HCV immunogen is an immunogenic HCV E1E2 complex with a contiguous sequence of amino acids having at least 80% sequence identity to the contiguous sequence of amino acids depicted at positions 192-809 of FIGS. 2A-2C, with the proviso that said polynucleotide does not encode an HCV immunogen other than the HCV E1E2 complex, wherein said HCV E1E2 complex is expressed in vivo to elicit an immune response.

8. The method of claim 7, wherein said HCV E1E2 complex consists of the sequence of amino acids depicted at positions 192-809 of FIGS. 2A-2C.

9. The method of claim 7, wherein the cationic microparticle is formed from a polymer selected from the group consisting of a poly(α-hydroxy acid), a polyhydroxy butyric acid, a polycaprolactone, a polyorthoester, and a polyanhydride.

10. The method of claim 9, wherein the cationic microparticle is formed from a poly(α-hydroxy acid) selected from the group consisting of poly(L-lactide), poly(D,L-lactide) and poly(D,L-lactide-co-glycolide).

11. The method of claim 10, wherein the cationic microparticle is formed from poly(D,L-lactide-co-glycolide).

12. The method of claim 7, further comprising administering a therapeutically effective amount of a second composition to the subject, wherein the second composition comprises an immunogenic HCV polypeptide and a pharmaceutically acceptable excipient.

13. The method of claim 12, wherein said second composition is administered subsequent to the first composition.

14. The method of claim 12, wherein said immunogenic HCV polypeptide in said second composition is an immunogenic HCV E1E2 complex with a contiguous sequence of amino acids having at least 80% sequence identity to the contiguous sequence of amino acids depicted at positions 192-809 of FIGS. 2A-2C.

15. The method of claim 14, wherein said HCV E1E2 complex consists of the sequence of amino acids depicted at positions 192-809 of FIGS. 2A-2C.

16. The method of claim 12, wherein said second composition further comprises an adjuvant.

17. The method of claim 16, wherein said adjuvant is a submicron oil-in-water emulsion capable of enhancing the immune response to the immunogenic HCV polypeptide, wherein the submicron oil-in-water emulsion comprises (i) a metabolizable oil, wherein the oil is present in an amount of 1% to 12% of the total volume, and (ii) an emulsifying agent, wherein the emulsifying agent is present in an amount of 0.01% to 1% by weight (w/v) and comprises polyoxyethylene sorbitan mono-, di-, or triester and/or a sorbitan mono-, di-, or triester, wherein the oil and the emulsifying agent are present in the form of an oil-in-water emulsion having oil droplets substantially all of which are about 100 nm to less than 1 micron in diameter.

18. The method of claim 17, wherein the submicron oil-in-water emulsion comprises 4-5% w/v squalene, 0.25-1.0% w/v polyoxyelthylenesorbitan monooleate, and/or 0.25-1.0% sorbitan trioleate, and optionally, N-acetylmuramyl-L-alanyl-D-isogluatminyl-L-alanine-2-(1′-2′-dipalmitoyl-sn-glycero-3-huydroxyphosphoryloxy)-ethylamine (MTP-PE).

19. The method of claim 17, wherein the submicron oil-in-water emulsion consists essentially of about 5% by volume of squalene; and one or more emulsifying agents selected from the group consisting of polyoxyelthylenesorbitan monooleate and sorbitan trioleate, wherein the total amount of emulsifying agent(s) present is about 1% by weight (w/v).

20. The method of claim 19, wherein the one or more emulsifying agents are polyoxyelthylenesorbitan monooleate and sorbitan trioleate and the total amount of polyoxyelthylenesorbitan monooleate and sorbitan trioleate present is about 1% by weight (w/v).

21. The method of claim 12, wherein said second composition further comprises a CpG oligonucleotide.

22. A method of stimulating an immune response in a vertebrate subject which comprises:
(a) administering to the subject a therapeutically effective amount of a first composition consisting essentially of a polynucleotide adsorbed to a cationic microparticle formed from poly(D,L-lactide-co-glycolide), wherein said polynucleotide comprises a coding sequence that encodes a hepatitis C virus (HCV) immunogen operably linked to control elements that direct the transcription and translation of said coding sequence in vivo, and further wherein the HCV immunogen is an HCV E1E2 complex consisting of the sequence of amino acids depicted at positions 192-809 of FIGS. 2A-2C, with the proviso that said polynucleotide does not encode an HCV immunogen other than the HCV E1E2 complex, and wherein said HCV E1E2 complex is expressed in vivo; and
(b) administering a therapeutically effective amount of a second composition to the subject, wherein the second composition comprises (i) an immunogenic HCV E1E2 complex consisting of the sequence of amino acids depicted at positions 192-809 of FIGS. 2A-2C, (ii) an adjuvant, and (iii) a pharmaceutically acceptable excipient, to elicit an immune response in the subject.

23. The method of claim 22, wherein said adjuvant is a submicron oil-in-water emulsion capable of enhancing the immune response to the immunogenic HCV E1E2 complex in the second composition, wherein the submicron oil-in-water emulsion comprises (i) a metabolizable oil, wherein the oil is present in an amount of 1% to 12% of the total volume, and (ii) an emulsifying agent, wherein the emulsifying agent is present in an amount of 0.01% to 1% by weight (w/v) and comprises polyoxyethylene sorbitan mono-, di-, or triester and/or a sorbitan mono-, di-, or triester, wherein the oil and the emulsifying agent are present in the form of an oil-in-water emulsion having oil droplets substantially all of which are about 100 nm to less than 1 micron in diameter.

24. The method of claim 23, wherein the submicron oil-in-water emulsion comprises 4-5% w/v squalene, 0.25-1.0% w/v polyoxyelthylenesorbitan monooleate, and/or 0.25-1.0% sorbitan trioleate, and optionally, N-acetylmuramyl-L-alanyl-D-isogluatminyl-L-alanine-2-(1′-2′-dipalmitoyl-sn-glycero-3-huydroxyphosphoryloxy)-ethylamine (MTP-PE).

25. The method of claim 23, wherein the submicron oil-in-water emulsion consists essentially of about 5% by volume of squalene; and one or more emulsifying agents selected from the group consisting of polyoxyelthylenesorbitan monooleate and sorbitan trioleate, wherein the total amount of emulsifying agent(s) present is about 1% by weight (w/v).

26. The method of claim 25, wherein the one or more emulsifying agents are polyoxyelthylenesorbitan monooleate and sorbitan trioleate and the total amount of polyoxyelthylenesorbitan monooleate and sorbitan trioleate present is about 1% by weight (w/v).

27. The method of claim 23, wherein said second composition further comprises a CpG oligonucleotide.

28. A method of making a composition comprising combining a pharmaceutically acceptable excipient with a polynucleotide adsorbed to a cationic microparticle, wherein said polynucleotide comprises a coding sequence that encodes a hepatitis C virus (HCV) immunogen operably linked to control elements that direct the transcription and translation of said coding sequence in vivo, and further wherein the HCV immunogen is an immunogenic HCV E1E2 complex with a contiguous sequence of amino acids having at least 80% sequence identity to the contiguous sequence of amino acids depicted at positions 192-809 of FIGS. 2A-2C, with the proviso that said polynucleotide does not encode an HCV immunogen other than the HCV E1E2 complex.

29. Use of a composition according to any of claims 1-6 in a method for stimulating an immune response in a vertebrate subject.
